(19) United States
(12) Patent Application Publication
(10) Pub. No.: US 2025/0026723 A1
(43) Pub. Date: Jan. 23, 2025

(54) Cannabinoid Receptor Modulating Compounds

(71) Applicant: THE UNITED STATES OF AMERICA, as represented by the Secretary, Department of Health and Human, Rockville, MD (US)

(72) Inventors: Malliga R. Iyer, Rockville, MD (US); George Kunos, Rockville, MD (US); Resat Cinar, Rockville, MD (US)

(21) Appl. No.: 18/709,949

(22) PCT Filed: Dec. 8, 2022
(86) PCT No.: PCT/US2022/081131

§ 371 Date: May 14, 2024

Related U.S. Application Data
(60) Provisional application No. 63/265,225, filed on Dec. 10, 2021.

(51) Int. Cl. C07D 231/06; C07D 211/96

(52) U.S. Cl. CPC C07D 231/06; C07D 211/96

(57) ABSTRACT

In accordance with the purpose(s) of the present disclosure, as embodied and broadly described herein, the disclosure, in one aspect, relates to sulfur- and selenium-containing compounds that act as agonists and/or antagonists of cannabinoid receptors, methods of making same, pharmaceutical compositions comprising the same, and methods of treating metabolic disorders, psychiatric disorders, neurological disorders, pain disorders, gastrointestinal disorders, cancers, inflammation-related disorders, substance abuse associated pathologies, and other conditions using the same.

### CROSS REFERENCE TO RELATED APPLICATIONS

This application claims the benefit of U.S. Provisional Application No. 63/265,225 filed on Dec. 10, 2021, which is incorporated herein by reference in its entirety.

### BACKGROUND

Cannabinoids are a class of secondary metabolites from plants in the Cannabis family (i.e., C. sativa, C. indica, and C. ruderalis) and are naturally formed through isoprenoid and polyketide synthesis pathways in these plants, with some modifications (e.g. methylation, acetylation, hydrogenation) occurring as a result of activity of other enzymes. Cannabis has a long history of both recreational and medicinal use and, with the legalization of marijuana in several states and countries, cannabinoids are increasingly being explored as alternatives for the treatment of a variety of diseases and conditions. However, levels of secondary metabolites in plants can be strongly dependent on cultivar, growing conditions, and the like, and many natural cannabinoids may not possess desired levels of solubility, stability, and/or other pharmacokinetic properties.

Two subtypes of cannabinoid receptors can be found in the human body. These are known as CB1, which is primarily expressed in the central nervous system as well as in the lungs, liver, and kidneys, and CB2, which is found in the immune system (e.g. T cells, macrophages, and B cells) and hematopoietic stem cells and, in some cases, in peripheral nerve terminals. CB1 is implicated in modulating several signaling pathways such as, for example, GABA- and glutamate-mediated neurotransmission, and may be involved in lipogenesis in the liver and adipose tissue, while CB2 is involved in anti-inflammatory effects and pain perception and relief and is further believed to possess several as-yet unidentified functions. Additional novel cannabinoid receptors GPR55, GPR119, and GPR18 have also been identified.

Current research has indicated that natural and synthetic or semi-synthetic cannabinoids may be effective treatments for numerous diseases and medical conditions including, but not limited to, metabolic disorders, psychiatric disorders, neurological disorders, pain disorders, gastrointestinal disorders, cancers, inflammation-related disorders, substance overuse associated pathologies, and other conditions. However, most studies have focused on the effects of Δ⁹-tetrahydrocannabinol (THC), cannabidiol (CBD), and combinations thereof.

Despite advances in cannabinoid research, there is still a scarcity of compounds that are potent, efficacious, and selective agonists or antagonists of CB1, CB2, GPR55, GPR18, GPR119, and other novel or putative cannabinoid receptors while also being effective in the treatment of various diseases and conditions. These needs and other needs are satisfied by the present disclosure.

### SUMMARY

In accordance with the purpose(s) of the present disclosure, as embodied and broadly described herein, the disclosure, in one aspect, relates to sulfur- and selenium-containing compounds that act as agonists and/or antagonists of cannabinoid receptors, methods of making same, pharmaceutical compositions comprising the same, and methods of treating metabolic disorders, psychiatric disorders, neurological disorders, pain disorders, gastrointestinal disorders, cancers, inflammation-related disorders, substance overuse associated pathologies, and other conditions using the same.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows an exemplary three-dimensional structure of a disclosed compound.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Disclosed herein are compounds having a structure of formula I,

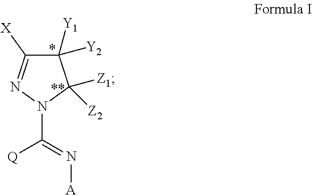

wherein X can be naphthyl, heteroaryl, linear or branched C₁-C₁₀ alkyl, or

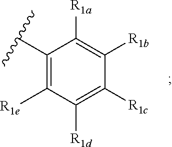

wherein each of R₁ₐ—R₁ₑ independently is selected from hydrogen, halogen, hydroxy, C₁-C₆ alkyl, —CF₃, —OCF₃, —O—(C₁-C₆ alkyl), —NO₂, or —CN;
wherein Y₁ and Y₂ independently can be hydrogen, naphthyl, heteroaryl, linear or branched C₁-C₁₀ alkyl, or

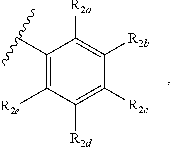

provided Y₁ and Y₂ are not both hydrogen;
wherein each of R₂ₐ—R₂ₑ independently is selected from hydrogen, halogen, hydroxy, C₁-C₆ alkyl, fluoroalkyl, fluoroalkoxy, —O—(C₁-C₆ alkyl), —NO₂, or —CN;
wherein Z₁ and Z₂ independently can be hydrogen, —CO(═O)—(C₁-C₆ alkyl), or —C(═O)NH—(C₁-C₆ alkyl);
wherein Q can be —S—R₃, —S(═O)—R₃, —S(═O)₂—R₃, —(CH₂)_{z}—R₃, —Se—R₃, —Se(═O)—R₃, or —Se(═O)₂—R₃, or OR₃;
wherein R₃ is selected from hydrogen, substituted or unsubstituted branched or linear C₁-C₆ alkyl, C₁-C₆ alkene, C₁-C₆ alkyne, C₁-C₆ allene, C₃-C₈ cycloalkyl, C₃-C₈ heterocycloalkyl, —(C(R₁₄ₐ)(R_{14b}))ₓ—C(═O)—N(R₁₃ₐ)(R_{13b}), —(C(R₁₄ₐ)(R_{14b}))_{y}—C(═O)—NHOH, or —(C(R₁₄ₐ)(R_{14b}))_{y}—CH(CH₃)—C(═O)(OR₁₀); —(CH₂)_{y}CN, —(CH₂)_{y}NO₂, —(CH₂)_{y}NHNH₂, —(C(R₁₄ₐ)(R_{14b}))_{y}SO₃R, —(C(R₁₄ₐ)(R_{14b}))_{y}N₃, —(C(R₁₄ₐ)(R_{14b}))_{y}—C₂H₂N₃, —(C(R₁₄ₐ)(R_{14b}))_{y}SON(R₁₃ₐ)(R_{13b}), —(C(R₁₄ₐ)(R_{14b}))_{y}SO₂N(R₁₃ₐ)(R_{13b}), —(C(R₁₄ₐ)(R_{14b}))_{y}PON(R₁₃ₐ)(R_{13b}), —(C(R₁₄ₐ)(R_{14b}))_{y}PO(OR₁₅)NR₁₆, —(C(R₁₄ₐ)(R_{14b}))_{y}B(OR₁₅)₃, —(C(R₁₄ₐ)(R_{14b}))_{y}(C═NR₁₆)N(R₁₃ₐ)(R_{13b}), C₁-C₆ alkoxy, OC(R₁₇)₃, OSi(R₁₇)₃, or —OCHF₂;
wherein x is from 1 to 6;
wherein y is from 0 to 6;
wherein R₁₀ is C₁-C₆ alkyl;
wherein each of R₁₃ₐ, R_{13b}, R₁₄ₐ, R_{14b}, R₁₅, R₁₆, and R₁₇, when present, is independently selected from hydrogen, halogen, hydroxy, C₁-C₆ alkyl, fluoroalkyl, fluoroalkoxy, —O—(C₁-C₆ alkyl), —NO₂, or —CN;
provided that when Q is —S—R₃, R₃ is not linear or branched alkyl;
wherein A can be —SO₂—R₄, —SeO₂—R₄—S(═O)—R₄, —S(═O)(—R₄)(—R₅), —S(═O)(═NH)—R₄, —C(═O)—R₄, —Se—R₄, —Se(═O)—R₄, —Se(═O)(—R₄)(—R₅), or —N(R₁₉ₐ)(R_{19b});
wherein R₄ is

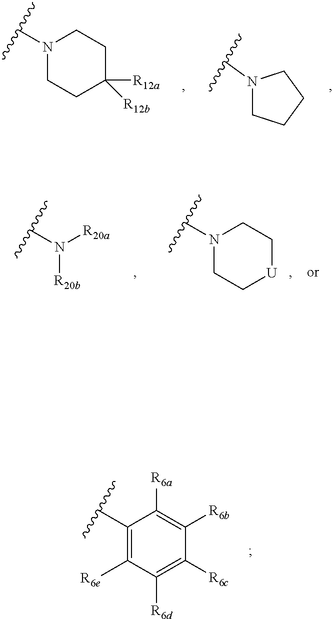

wherein each of R₁₉ₐ and R_{19b} is independently selected from hydrogen, halogen, hydroxy, substituted or unsubstituted C₁-C₆ alkyl, fluoroalkyl, fluoroalkoxy, —O—(C₁-C₆ alkyl), —NO₂, or —CN, and wherein R_{19b} is optionally absent;
wherein each of R₂₀ₐ and R_{20b} is independently selected from linear or branched C₁-C₆ alkyl or adamantyl;
   wherein each of R₁₂ₐ and R_{12b} independently is hydrogen, CF₃, phenyl, fluoro, or any combination thereof;
wherein U is NR₁₃, S, SO₂, or O;
   wherein R₁₈ is selected from hydrogen, halogen, hydroxy, C₁—C alkyl, fluoroalkyl, fluoroalkoxy, —O—(C₁-C₆ alkyl), —NO₂, or —CN;
wherein each of R₆ₐ—R₆ₑ independently is selected from hydrogen, halogen, hydroxy, C₁-C₆ alkyl, fluoroalkyl, fluoroalkoxy, —O—(C₁-C₆ alkyl), —NO₂, —CN, —(CH₂)_{z}—R₇, naphthyl, or heteroaryl;
wherein R₇ can be halogen, —CN, fluoroalkyl, fluoroalkoxy, or alkoxy;
wherein z is from 1 to 3;
wherein R₅ can be (═O), —NH—R₃, or

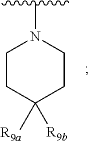

wherein R₃ is hydrogen or C₁-C₆ alkyl; and
wherein each of R₉ₐ and R_{9b} independently can be hydrogen or halogen.

In one aspect, the compounds can have (R) or (S) stereochemistry or can be diastereomers. Also disclosed are methods of making the compounds and pharmaceutical compositions including the compounds and methods of treating one or more metabolic disorders, psychiatric disorders, neurological disorders, pain disorders, gastrointestinal disorders, cancers, inflammatory disorders, liver diseases, cardiovascular disorders, a disease or condition associated with abnormal histone deacetylase activity, or substance overuse associated pathologies in a subject, the method including the step of administering to the subject a therapeutically effective amount of the compounds or the pharmaceutical compositions.

In an aspect, the disclosed compounds can be functional agonists, neutral or inverse antagonists, allosteric modulators, biased modulators (e.g., acting specifically via G-protein or p-arrestin), or any combination thereof, with respect to one or more cannabinoid receptors or cannabinoid receptor analogues. In a further aspect, the disclosed compounds can be brain penetrant or peripherally restricted. In a still further aspect, when the compounds are brain penetrant, the compounds can have features that can abrogate or minimize psychotropic or anxiogenic effects.

In another aspect, the compounds can be used alone or in combination with experimental or approved drugs for treatment of the aforementioned diseases and disorders. Further in this aspect, the compounds can be used in combination with GLP-1 agonists, integrin inhibitors, galectin inhibitors, CB2 agonists, CB1 antagonists, metformin and other AMPK activators, mGlur5 antagonists, 5HT2B antagonists, iNOS inhibitors, SSAO/VAP-1 inhibitors, and combinations thereof.

In another aspect, the compounds can have unique fluorescence properties and may be of use as probes in imaging applications as well as environmental applications. In one aspect, imaging applications and environmental probes can include affinity labeling, biotinylation, photochemical probes, and the like.

In one aspect, Q can be useful for introducing a secondary pharmacophore or fragment that could interact with one or more other targets in addition to CB1, CB2, GPR55, GPR18, and/or GPR119. In one aspect, other targets and processes can include but are not limited to inducible nitric oxide synthase, histamine modulation, proton pump inhibition, histone deacetylase HDAC inhibition, and the like. In any of these aspects, the disclosed compounds may be useful in Pharmacophore Assisted Novel Targeting of Multi-Mechanisms (PhaNToMM), Fragment Aided Novel Discovery of Multi-Mechanisms (FaNDoMM), or any combination thereof. In one aspect, on a main privileged scaffold, a pharmacophore or fragment can be attached such that this fragment can modulate a target which can synergize or complement additively to the main scaffold (such as, for example, CB1) to increase/improve efficacy. Several non-limiting examples of pharmacophores and/or fragments are shown below (boxed elements):

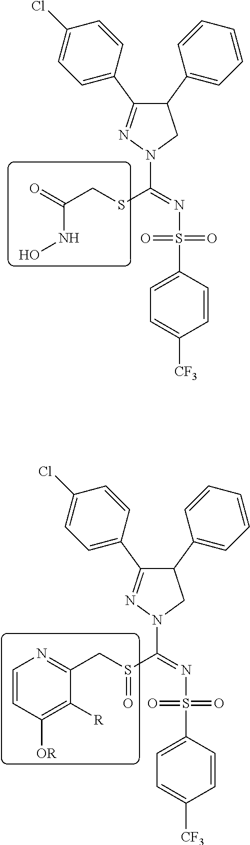

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C6 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkanediyl” as used herein, refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, and —CH₂CH₂CH₂— are non-limiting examples of alkanediyl groups.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A² where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) and —N(-alkyl)₂, where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen” or “halide,” as used herein can be used interchangeably and refer to F, Cl, Br, or I.

The terms “pseudohalide,” “pseudohalogen” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1 H-pyrazolo[4,3-c]pyridin-3-yl; 1 H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxy” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” . . . “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R○; —(CH₂)₀₋₄OR○; —O(CH₂)₀₋₄R○, —O—(CH₂)₀₋₄C(O)OR○; —(CH₂)₀₋₄CH(OR○)₂; —(CH₂)₀₋₄SR○; —(CH₂)₀₋₄Ph, which may be substituted with R○; —(CH₂)₀₋₄O(CH₂)₀₋₁Ph which may be substituted with R○; —CH═CHPh, which may be substituted with R○; —(CH₂)₀₋₄O(CH₂)₀₋₁-pyridyl which may be substituted with R○; —NO₂; —CN; —N₃; —(CH₂)₀₋₄N(R○)₂; —(CH₂)₀₋₄N(R○)C(O)R○; —N(R○)C(S)R○; —(CH₂)₀₋₄N(R○)C(O)NR○₂; —N(R○)C(S)NR○₂; —(CH₂)₀₋₄N(R○)C(O)OR○; —N(R○)N(R○)C(O)R○; —N(R○)N(R○)C(O)NR○₂; —N(R○)N(R○)C(O)OR○; —(CH₂)₀₋₄C(O)R○; —C(S)R○; —(CH₂)₀₋₄C(O)OR○; —(CH₂)₀₋₄C(O)SR○; —(CH₂)₀₋₄C(O)OSiR○₃; —(CH₂)₀₋₄OC(O)R○; —OC(O)(CH₂)₀₋₄SR—, SC(S)SR○; —(CH₂)₀₋₄SC(O)R○; —(CH₂)₀₋₄C(O)NR○₂; —C(S)NR○₂; —C(S)SR○; —(CH₂)₀₋₄OC(O)NR○₂; —C(O)N(OR○)R○; —C(O)C(O)R○; —C(O)CH₂C(O)R○; —C(NOR○)R○; —(CH₂)₀₋₄SSR○; —(CH₂)₀₋₄S(O)₂R○; —(CH₂)₀₋₄S(O)₂OR○; —(CH₂)₀₋₄OS(O)₂R○; —S(O)₂NR○₂; —(CH₂)₀₋₄S(O)R○; —N(R○)S(O)₂NR○₂; —N(R○)S(O)₂R○; —N(OR○)R○; —C(NH)NR○₂; —P(O)₂R○; —P(O)R○₂; —OP(O)R○₂; —OP(O)(OR○)₂; SiR○₃; —(C₁₋₄ straight or branched alkylene)O—N(R○)₂; or —(C₁₋₄ straight or branched alkylene)C(O)O—N(R○)₂, wherein each R○ may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R○, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R○ (or the ring formed by taking two independent occurrences of R○ together with their intervening atoms), are independently halogen, —(CH₂)₀₋₂R●, -(haloR●), —(CH₂)₀₋₂OH, —(CH₂)₀₋₂OR●, —(CH₂)₀₋₂CH(OR●)₂; —O(haloR●), —CN, —N₃, —(CH₂)₀₋₂C(O)R●, —(CH₂)₀₋₂C(O)OH, —(CH₂)₀₋₂C(O)OR●, —(CH₂)₀₋₂SR●, —(CH₂)₀₋₂SH, —(CH₂)₀₋₂NH₂, —(CH₂)₀₋₂NHR●, —(CH₂)₀₋₂NR●₂, —NO₂, —SiR●₃, —OSiR●₃, —C(O)SR●, —(C₁₋₄ straight or branched alkylene)C(O)OR●, or —SSR● wherein each R● is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R○ include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R● include halogen, —R●, -(haloR●), —OH, —OR●, —O(haloR●), —CN, —C(O)OH, —C(O)OR●, —NH₂, —NHR●, —NR●₂, or —NO₂, wherein each R● is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R†, —NR†₂, —C(O)R†, —C(O)OR†, —C(O)C(O)R†, —C(O)CH₂C(O)R†, —S(O)₂R†, —S(O)₂NR†₂, —C(S)NR†₂, —C(NH)NR†₂, or —N(R†)S(O)₂R†; wherein each R† is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R†, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R† are independently halogen, —R^{●}, -(haloR●), —OH, —OR●, —O(haloR●), —CN, —C(O)OH, —C(O)OR●, —NH₂, —NHR●, —NR [symbol] ₂, or —NO₂, wherein each R● is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

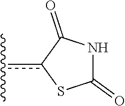

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms, and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers and tautomers (e.g., thiol/thiourea), as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and I or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine, chlorine, sulfur, selenium, tellurium, and iodine, such as ²H, ³H, ¹³C, ¹⁴C ¹⁵N, ¹⁸O, ¹⁷O, ³⁵S, ⁷⁴Se, ⁷⁶Se, ⁷⁷Se, ⁷⁸Se, ⁸⁰Se, ¹²²Te, ¹²³Te, ¹²⁴Te, ¹²⁵Te, ¹²⁶Te ¹⁸F, ³⁶Cl, ¹²⁴I, ¹²⁵I, and ¹³¹I, respectively. Compounds further comprise prodrugs thereof and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

It is also appreciated that certain compounds described herein can be present as an equilibrium of tautomers. For example, ketones with an α-hydrogen can exist in an equilibrium of the keto form and the enol form.

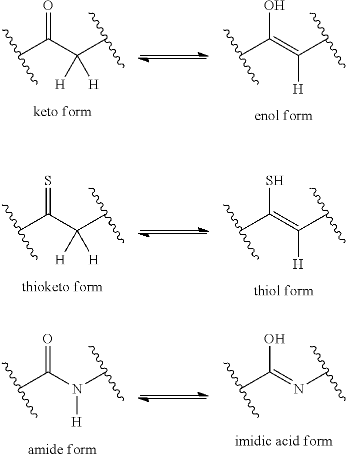 [SMILES: *CC(*)=O ; */C=C(/*)O ; *CC(*)=S ; */C=C(/*)S ; *NC(*)=O ; */N=C(/*)O]

Likewise, amides with an N-hydrogen can exist in an equilibrium of the amide form and the imidic acid form, and thiols can exist in a thiol/thioketo equilibrium, and the like. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

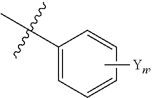

which is understood to be equivalent to a formula:

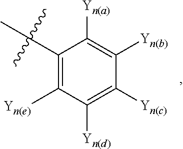

wherein n is typically an integer. That is, Yₙ is understood to represent five independent substituents, Yₙ₍ₐ), Y_{n(b)}, Y_{n(c)}, Y^{n(d)}, and Yₙ₍ₑ₎. By “independent substituents,” it is meant that each Y substituent can be independently defined. For example, if in one instance Yₙ₍ₐ₎ is halogen, then Y_{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B—F, C-D, C-E, and C—F are considered disclosed. Likewise, any subset or combination of these is also disclosed. Thus, for example, the sub-group of A-E, B—F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a cannabinoid receptor modulating compound,” “a solvent,” or “a source of chloride,” includes, but is not limited to, mixtures or combinations of two or more such cannabinoid receptor modulating compounds, solvents, or sources of chloride, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

In one aspect, with reference to a single enantiomer or mixture of enantiomers, or a diastereomer or mixture of diastereomers, “substantially” can be used to describe a greater than 50% enantiomeric excess (ee) of either an R or an S enantiomer at a given chiral carbon, wherein R or S stereochemistry is determined by the Cahn-Ingold-Prelog system. In another aspect, “substantially” can be used to describe greater than 60% ee, greater than 70% ee, greater than 80% ee, greater than 90% ee, greater than 95% ee, or about 100% ee, or a combination of any of the foregoing values, or a range encompassing any of the foregoing values.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### Cannabinoid Receptor Modulating Compounds

In one aspect, disclosed herein is a compound having a structure of Formula I,

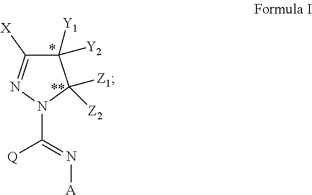

wherein X can be naphthyl, heteroaryl, linear or branched C₁-C₁₀ alkyl, or

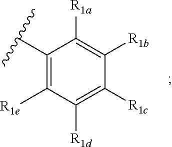

wherein each of R₁ₐ—R₁ₑ independently is selected from hydrogen, halogen, hydroxy, C₁-C₆ alkyl, —CF₃, —OCF₃, —O—(C₁-C₆ alkyl), —NO₂, or —CN;
wherein Y₁ and Y₂ independently can be hydrogen, naphthyl, heteroaryl, linear or branched C₁-C₁₀ alkyl, or

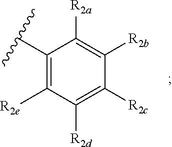

provided Y₁ and Y₂ are not both hydrogen;
wherein each of R₂ₐ—R₂ₑ independently is selected from hydrogen, halogen, hydroxy, C₁-C₆ alkyl, fluoroalkyl, fluoroalkoxy, —O—(C₁-C₆ alkyl), —NO₂, or —CN;
wherein Z₁ and Z₂ independently can be hydrogen, —CO(═O)—(C₁-C₆ alkyl), or —C(═O)NH—(C₁-C₆ alkyl);
wherein Q can be —S—R₃, —S(═O)—R₃, —S(═O)₂—R₃, —(CH₂)_{z}—R₃, —Se—R₃, —Se(═O)—R₃, or —Se(═O)₂—R₃, or OR₃;
wherein R₃ is selected from hydrogen, substituted or unsubstituted branched or linear C₁-C₆ alkyl, C₁-C₆ alkene, C₁-C₆ alkyne, C₁-C₆ allene, C₃-C₈ cycloalkyl, C₃-C₈ heterocycloalkyl, —(C(R₁₄ₐ)(R_{14b}))ₓC(═O)—N(R₁₃ₐ)(R_{13b}), —(C(R₁₄ₐ)(R_{14b}))_{y}—C(═O)—NHOH, or —(C(R₁₄ₐ)(R_{14b}))_{y}—CH(CH₃)—C(═O)(OR₁₀); —(CH₂)_{y}CN, —(CH₂)_{y}NO₂, —(CH₂)_{y}NHNH₂, —(C(R₁₄ₐ)(R_{14b}))_{y}SO₃R, —(C(R₁₄ₐ)(R_{14b}))_{y}N₃, —(C(R₁₄ₐ)(R_{14b}))_{y}—C₂H₂N₃, —(C(R₁₄ₐ)(R_{14b}))_{y}SON(R₁₃ₐ)(R_{13b}), —(C(R₁₄ₐ)(R_{14b}))_{y}SO₂N(R₁₃ₐ)(R_{13b}), —(C(R₁₄ₐ)(R_{14b}))_{y}PON(R₁₃ₐ)(R_{13b}), —(C(R₁₄ₐ)(R_{14b}))_{y}PO(OR₁₅)NR₁₆, —(C(R₁₄ₐ)(R_{14b}))_{y}B(OR₁₅)₃, —(C(R₁₄ₐ)(R_{14b}))_{y}(C═NR₁₆)N(R₁₃ₐ)(R_{13b}), C₁-C₆ alkoxy, OC(R₁₇)₃, OSi(R₁)₃, or —OCHF₂;
wherein x is from 1 to 6;
wherein y is from 0 to 6;
   wherein R₁₀ is C₁-C₆ alkyl;
   wherein each of R₁₃ₐ, R_{13b}, R₁₄ₐ, R_{14b}, R₁₅, R₁₆, and R₁₇, when present, is independently selected from hydrogen, halogen, hydroxy, C₁-C₆ alkyl, fluoroalkyl, fluoroalkoxy, —O—(C₁-C₆ alkyl), —NO₂, or —CN;
provided that when Q is —S—R₃, R₃ is not linear or branched alkyl;
wherein A can be —SO₂—R₄, —SeO₂—R₄—S(═O)—R₄, —S(═O)(—R₄)(—R₅), —S(═O)(═NH)—R₄, —C(═O)—R₄, —Se—R₄, —Se(═O)—R₄, —Se(═O)(—R₄)(—R₅), or —N(R₁₉ₐ)(R_{19b});
wherein R₄ is

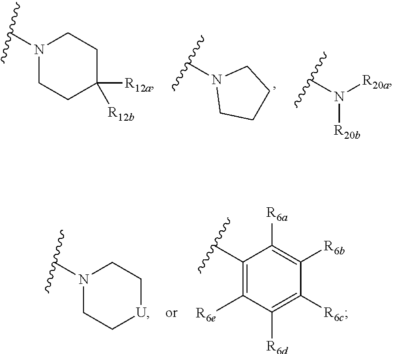

wherein each of R₁₉ₐ and R_{19b} is independently selected from hydrogen, halogen, hydroxy, substituted or unsubstituted C₁-C₆ alkyl, fluoroalkyl, fluoroalkoxy, —O—(C₁-C₆ alkyl), —NO₂, or —CN, and wherein R_{19b} is optionally absent;
wherein each of R₂₀ₐ and R_{20b} is independently selected from linear or branched C₁-C₆ alkyl or adamantyl;
wherein each of R₁₂ₐ and R_{12b} independently is hydrogen, CF₃, phenyl, fluoro, or any combination thereof;
   wherein U is NR₁₈, S, SO₂, or O;
      wherein R₁₈ is selected from hydrogen, halogen, hydroxy, C₁-C₆ alkyl, fluoroalkyl, fluoroalkoxy, —O—(C₁-C₆ alkyl), —NO₂, or —CN;
wherein each of R₆ₐ—R₆ₑ independently is selected from hydrogen, halogen, hydroxy, C₁-C₆ alkyl, fluoroalkyl, fluoroalkoxy, —O—(C₁-C₆ alkyl), —NO₂, —CN, —(CH₂)_{z}—R₇, naphthyl, or heteroaryl;
wherein R₇ can be halogen, —CN, fluoroalkyl, fluoroalkoxy, or alkoxy;
wherein z is from 1 to 3;
wherein R₅ can be (═O), —NH—R₃, or

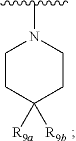

wherein R₃ is hydrogen or C₁-C₆ alkyl; and
wherein each of R₉ₐ and R_{9b} independently can be hydrogen or halogen.

In another aspect, in Formula I, X is

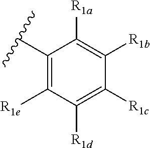

and each of R₁ₐ—R₁ₑ is independently —CF₃, —OCF₃, or hydrogen, or X is

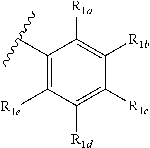

and each of R₁ₐ—R₁ₑ is independently hydrogen or halogen, or X can be

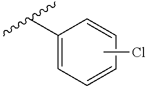

or can be

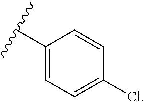 [SMILES: *c1ccc(Cl)cc1]

In one aspect, R₃ is —(CH₂)ₙ—R₁₁; n is from 1 to 6; and R₁₁ is cyano, methoxy, halogen, —CF₃, —OCF₃, nitro, or any combination thereof. In another aspect, R₃ is —(CH₂)₃—CN, —(CH₂)₂—OCH₃, or any combination thereof.

In still another aspect, R₄ can be

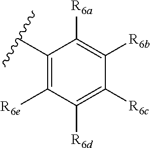

and each of R₆ₐ—R_{6c} is independently —CF₃, —OCF₃, or hydrogen.

In a further aspect, R₇ can be —CF₃, —OCF₃, or methoxy.

In still another aspect, in Formula I, Y₁ is hydrogen and Y₂ is

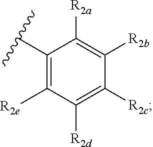

and each of R₂ₐ—R₂ₑ is independently hydrogen or halogen. In some aspects, Y₂ is phenyl.

In another aspect, in the compounds of Formula I, the compounds can have a substantially (R) stereochemistry or a substantially (S) stereochemistry at the carbon indicated by *.

In one aspect, in Formula I, Z₁ and Z₂ are hydrogen, or one of Z₁ or Z₂ is not hydrogen and the compound has a substantially (R) stereochemistry or a substantially (S) stereochemistry at the carbon indicated by **.

In still another aspect, Q can be selected from —S—R₃, —S(═O)—R₃, and —S(═O)₂—R₃. In another aspect, R₃ can be selected from

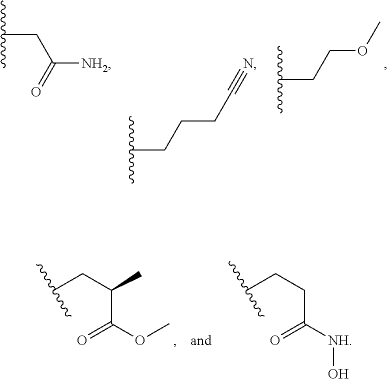

In still another aspect, A is —S—R₄, —S(═O)—R₄, or —S(═O)₂—R₄, while R₄ is selected from

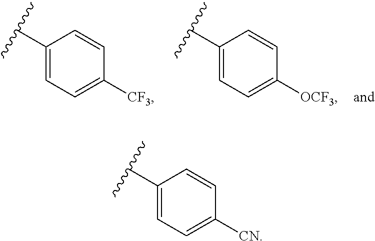 [SMILES: *c1ccc(C(F)(F)F)cc1 ; *c1ccc(OC(F)(F)F)cc1 ; *c1ccc(C#N)cc1]

In one aspect, in Formula I, X is

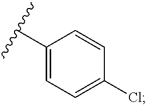 [SMILES: *c1ccc(Cl)cc1]

Y₁ is hydrogen and Y₂ is phenyl; Z₁ and Z₂ are hydrogen; Q is selected from —S—R₃, —S(═O)—R₃, and —S(═O)₂—R₃ and R₃ is

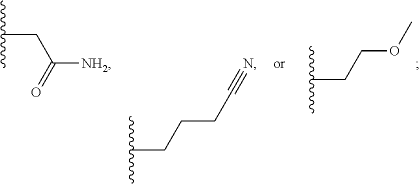

and A is —S—R₄, —S(═O)—R₄, or —S(═O)₂—R₄, wherein R₄ is

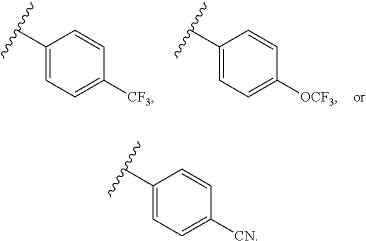 [SMILES: *c1ccc(C(F)(F)F)cc1 ; *c1ccc(OC(F)(F)F)cc1 ; *c1ccc(C#N)cc1]

In yet another aspect, the compound is

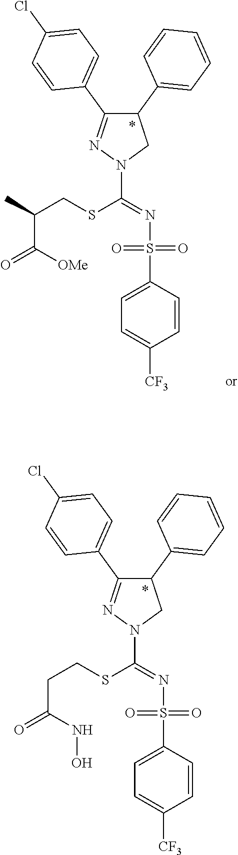 [SMILES: COC(=O)[C@@H](C)CS/C(=N\S(=O)(=O)c1ccc(C(F)(F)F)cc1)N1CC(c2ccccc2)C(c2ccc(Cl)cc2)=N1 ; O=C(CCS/C(=N\S(=O)(=O)c1ccc(C(F)(F)F)cc1)N1CC(c2ccccc2)C(c2ccc(Cl)cc2)=N1)NO]

and has a substantially (S) stereochemistry at a carbon indicated by *.

In another aspect, disclosed herein are bivalent and/or multivalent compounds and compositions including two or more of the disclosed compounds, or including two or more of the same compound joined by a linking group, wherein the linking group can be positioned at various sites on the disclosed scaffolds.

In any of these aspects, the compounds bind to cannabinoid receptor 1 (CB1), cannabinoid receptor 2 (CB2), GPR55, GPR18, GPR119, or any combination thereof. In a further aspect, the compound has a CB1 inhibition constant (Kᵢ) of less than about 100 nM, or less than about 75, 50, or 25 nM, or a combination of any of the foregoing values, or a range encompassing any of the foregoing values In an alternative aspect, the compound has a CB2 inhibition constant (Kᵢ) of less than about 100 nM.

### Methods of Making the Compounds

Exemplary methods for synthesizing the compounds disclosed herein are summarized in Scheme 1, with details being provided below and wherein X, Y₁, Y₂, Z₁, Z₂, R₃, and R₄ are defined as above.

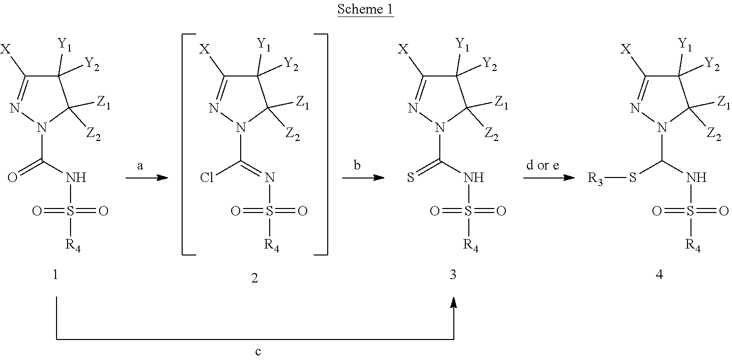

### Method 1

In one aspect, disclosed herein is a method for synthesizing a compound of Formula I, the method including at least the following steps:
(a) admixing a starting material with a source of a leaving group to produce a first composition; and
(b) admixing the first composition with a compound having the formula M₂XYO₃·mH₂O to produce the compound of Formula I;
wherein M is selected from K, Na, or Li;
wherein X and Y are independently selected from S or Se; and
wherein m is from 0 to 6.

In one aspect, the leaving group can be a halide. In another aspect, the halide can be chloride, bromide, or iodide.

In one aspect, the source of leaving group can be a halogenating agent. In another aspect, many halogenating agents are known in the art and can be used in the practice of the disclosed methods. In another aspect, these include, but are not limited to, dialkyl and/or diaryl chloroiminium ion compounds, phosgene, oxalyl dichloride, thionyl chloride, phosphorus pentachloride, phosphorous trichloride, phosphorus oxychloride, carbonyl dibromide, oxalyl bromide, thionyl bromide, phosphorous bromide, and phosphorus oxybromide. In another aspect, halogenating agents can be used alone or in combination.

In one aspect, the halogenating agent can be a phosphorus halide having the formula PW₃, POW₃, PW₅, and wherein W is selected from Cl, Br, or I. In one aspect, the source of leaving group can be a phosphorus chloride compound such as, for example, PCl₅ or POCl₃. In some aspects, step (a) further includes admixing a base with the compound of Formula I and the source of a leaving group. In one aspect, the base can be N,N-diisopropylethylamine (DIPEA), triethylamine, imidazole, benzimidazole, guanidine, ammonium hydroxide, pyridine, sodium hydroxide, potassium hydroxide, 4-dimethylaminopyridine (DMAP), 1,8-diazabicyclo[5.4.0]undec-7-ene (DBU), or any combination thereof.

In any of these aspects, step (a) can be carried out in a solvent such as, for example, toluene, chlorobenzene, dimethylformamide, dichloromethane, xylenes, or any combination thereof.

In an aspect, the compound having the formula M₂XYO₃·mH₂O can be Na₂S₂O₃·5H₂O, Na₂S₂O₃, K₂S₂₀₃, K₂Se₂O₃, or K₂SeSO₃. In one aspect, X and Y can independently be selected from sulfur and selenium. In one aspect, X and/or Y can be sulfur and the compound having the formula M₂XYO₃·mH₂O can be Na₂S₂O₃ or K₂S₂O₃. In another aspect, X and/or Y can be selenium and the compound having the formula M₂XYO₃·mH₂O can be K₂Se₂O₃.

In some aspects, step (b) can further include admixing an additive with the compound of Formula II and the compound having the formula M₂XYO₃·mH₂O. In one aspect, the additive can be an organic or inorganic base including, but not limited to, triethylamine, Li₂CO₃, Na₂CO₃, K₂CO₃, MgCO₃, CaCO₃, BaCO₃, LiHCO₃, NaHCO₃, KHCO₃, Mg(HCO₃)₂, Ca(HCO₃)₂, tetrabutylammonium bromide, tetramethylammonium chloride, or any combination thereof. In another aspect, step (b) can be carried out in a solvent such as, for example, methanol, water, ethanol, dioxane, tetrahydrofuran, dimethylformamide, acetone, acetonitrile, DMSO, toluene, isopropyl alcohol, xylenes, ethylene glycol, or any combination thereof.

In any of these aspects, the compound having the formula M₂XYO₃·mH₂O, the compound of Formula I, and/or any other reactant or product are not malodorous.

### Method 2

In one aspect, disclosed herein is an alternate method for making a compound of Formula 1. In a further aspect, this method consists of a single step. In a still further aspect, a thionation reagent such as, for example, Lawesson's reagent, P₂S₅, or P₄S₁₀ can used to convert compound 1 from Scheme 1 directly to compound 3.

### Functionalization of Sulfinyl Group

In a still further aspect, following the synthesis of compound 3, the sulfinyl group can be modified to provide additional functionalities as in compound 4. In one aspect, in a first functionalization method, R₃-LG can be used to contact compound 3 in a solvent such as, for example, refluxing methanol, where LG is a leaving group and R₃ is defined as above. In another aspect, the leaving group (LG) can be I, Br, Cl, HSO₄⁻, tosylate, or another leaving group. In an alternative aspect, in a second functionalization method, R₃-LG can be used to contact compound 3 in the presence of a base in a solvent. In an aspect, the base can be an organic or inorganic base including, but not limited to, triethylamine, Li₂CO₃, Na₂CO₃, K₂CO₃, MgCO₃, CaCO₃, BaCO₃, LiHCO₃, NaHCO₃, KHCO₃, Mg(HCO₃)₂, Ca(HCO₃)₂, tetrabutylammonium bromide, tetramethylammonium chloride, or any combination thereof. In another aspect, the solvent can be methanol, water, ethanol, dioxane, tetrahydrofuran, dimethylformamide, acetone, acetonitrile, DMSO, toluene, isopropyl alcohol, xylenes, ethylene glycol, or any combination thereof. In one aspect, the base is K₂CO₃ and the solvent is DMF.

### Additional Modifications

In one aspect, the compounds synthesized by either method can be further modified by techniques known in the art. In one aspect, additional substituents can be employed at variable positions in order to impair or restrict brain penetration of the compounds.

### Method 3

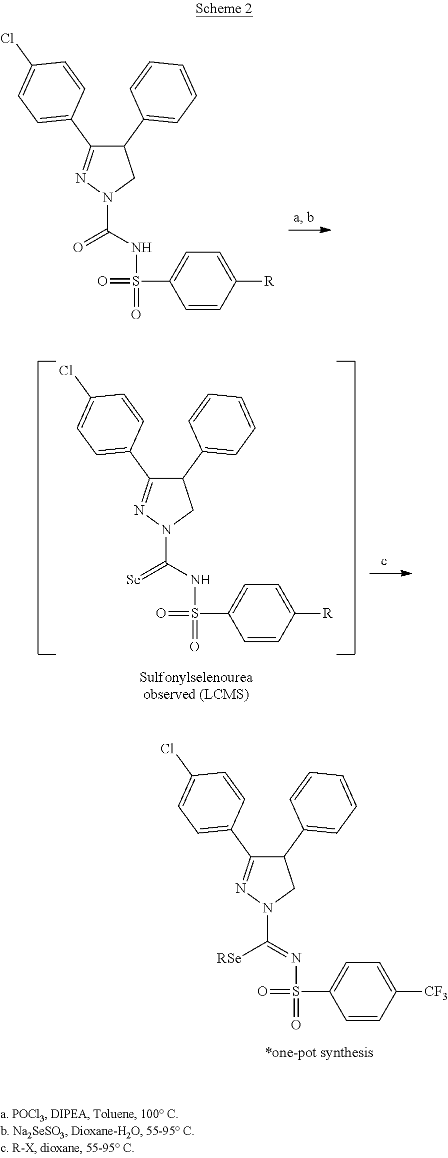

A general one-pot procedure for producing sulfonylselenoureas is shown in Scheme 2. By using sulfur containing salts in step (b) (e.g. Na₂S₂SO₃), analogous thiosulfonylureas can be produced. Exemplary reactions and synthetic work-up for one-pot thiosulfonylurea and/or sulfonylselenylurea formation and alkylation are provided in the Examples.

### Pharmaceutical Compositions

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom, or condition thereof, such as a cancer, pain, and/or weight loss associated with cancer therapy. The effect can be therapeutic in terms of a partial or complete cure of a disease, condition, symptom, or adverse effect attributed to the disease, disorder, or condition. The term “treatment” as used herein can include any treatment of a disease or condition in a subject in which CB1, CB2, GPR55, GPR18, and/or GPR119 activity is involved, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder, or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C 1-to-C 6 alkyl esters and C 5-to-C 7 cycloalkyl esters, although C 1-to-C 4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C 1-to-C 6 alkyl amines and secondary C 1-to-C 6 dialkyl amines. In the case of secondary amines, the amine can also be in the form of a 5- or 6-membered heterocycle containing one nitrogen atom. Amides derived from ammonia, C 1-to-C 3 alkyl primary amides and C 1-to-C 2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

The term “contacting” as used herein refers to bringing a disclosed compound or pharmaceutical composition in proximity to a cell, a target protein, or other biological entity together in such a manner that the disclosed compound or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitoneally, intraventricularly, intracranially, and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

Pharmaceutically acceptable salts can be prepared from pharmaceutically acceptable non-toxic bases or acids. For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In various aspects, a disclosed compound comprising an acidic group or moiety, e.g., a carboxylic acid group, can be used to prepare a pharmaceutically acceptable salt. For example, such a disclosed compound may comprise an isolation step comprising treatment with a suitable inorganic or organic base. In some cases, it may be desirable in practice to initially isolate a compound from the reaction mixture as a pharmaceutically unacceptable salt and then simply convert the latter back to the free acid compound by treatment with an acidic reagent, and subsequently convert the free acid to a pharmaceutically acceptable base addition salt. These base addition salts can be readily prepared using conventional techniques, e.g., by treating the corresponding acidic compounds with an aqueous solution containing the desired pharmacologically acceptable cations and then evaporating the resulting solution to dryness, preferably under reduced pressure. Alternatively, they also can be prepared by mixing lower alkanolic solutions of the acidic compounds and the desired alkali metal alkoxide together, and then evaporating the resulting solution to dryness in the same manner as before.

Bases which can be used to prepare the pharmaceutically acceptable base-addition salts of the base compounds are those which can form non-toxic base-addition salts, i.e., salts containing pharmacologically acceptable cations such as, alkali metal cations (e.g., lithium, potassium and sodium), alkaline earth metal cations (e.g., calcium and magnesium), ammonium or other water-soluble amine addition salts such as N-methylglucamine-(meglumine), lower alkanolammonium and other such bases of organic amines. In a further aspect, derived from pharmaceutically acceptable organic non-toxic bases include primary, secondary, and tertiary amines, as well as cyclic amines and substituted amines such as naturally occurring and synthesized substituted amines. In various aspects, such pharmaceutically acceptable organic non-toxic bases include, but are not limited to, ammonia, methylamine, ethylamine, propylamine, isopropylamine, any of the four butylamine isomers, betaine, caffeine, choline, dimethylamine, diethylamine, diethanolamine, dipropylamine, diisopropylamine, di-n-butylamine, N,N′-dibenzylethylenediamine, pyrrolidine, piperidine, morpholine, trimethylamine, triethylamine, tripropylamine, tromethamine, 2-diethylaminoethanol, 2-dimethylaminoethanol, ethanolamine, quinuclidine, pyridine, quinoline and isoquinoline; benzathine, N-methyl-D-glucamine, ethylenediamine, N-ethylmorpholine, N-ethylpiperidine, glucamine, glucosamine, methylglucamine, morpholine, piperazine, piperidine, polyamine resins, procaine, purines, theobromine, hydrabamine salts, and salts with amino acids such as, for example, histidine, arginine, lysine and the like. The foregoing salt forms can be converted by treatment with acid back into the free acid form.

In various aspects, a disclosed compound comprising a protonatable group or moiety, e.g., an amino group, can be used to prepare a pharmaceutically acceptable salt. For example, such a disclosed compound may comprise an isolation step comprising treatment with a suitable inorganic or organic acid. In some cases, it may be desirable in practice to initially isolate a compound from the reaction mixture as a pharmaceutically unacceptable salt and then simply convert the latter back to the free base compound by treatment with a basic reagent, and subsequently convert the free base to a pharmaceutically acceptable acid addition salt. These acid addition salts can be readily prepared using conventional techniques, e.g., by treating the corresponding basic compounds with an aqueous solution containing the desired pharmacologically acceptable anions and then evaporating the resulting solution to dryness, preferably under reduced pressure. Alternatively, they also can be prepared by treating the free base form of the disclosed compound with a suitable pharmaceutically acceptable non-toxic inorganic or organic acid.

Acids that can be used to prepare the pharmaceutically acceptable acid-addition salts of the base compounds are those which can form non-toxic acid-addition salts, i.e., salts containing pharmacologically acceptable anions formed from their corresponding inorganic and organic acids. Exemplary, but non-limiting, inorganic acids include hydrochloric hydrobromic, sulfuric, nitric, phosphoric and the like. Exemplary, but non-limiting, organic acids include acetic, benzenesulfonic, benzoic, camphorsulfonic, citric, ethanesulfonic, fumaric, gluconic, glutamic, isethionic, lactic, maleic, malic, mandelicmethanesulfonic, mucic, pamoic, pantothenic, succinic, tartaric, p-toluenesulfonic acid and the like. In a further aspect, the acid-addition salt comprises an anion formed from hydrobromic, hydrochloric, maleic, phosphoric, sulfuric, and tartaric acids.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets, or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs, and solutions; while carriers such as starches, sugars, microcrystalline cellulose, diluents, granulating agents, lubricants, binders, disintegrating agents, and the like can be used to form oral solid preparations such as powders, capsules, and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentaerythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid, or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate, and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate, and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, Iinolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants, and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions, or suspensions, including saline and buffered media. Parenteral vehicles can include mannitol, normal serum albumin, sodium chloride solution, Ringer's dextrose, dextrose and sodium chloride, lactated Ringer's, and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus, and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin, and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier, and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic, or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin, and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container, and labeled for treatment of an indicated condition.

Another patch system configuration which can be used by the present invention is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present invention is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In the treatment conditions which require modulation of CB1, CB2, GPR55, GPR18, and/or GPR119 activity, an appropriate dosage level will generally be about 0.01 to 1000 mg per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The disclosed pharmaceutical compositions can further comprise other therapeutically active compounds, which are usually applied in the treatment of the above mentioned pathological or clinical conditions.

It is understood that the disclosed compositions can be prepared from the disclosed compounds. It is also understood that the disclosed compositions can be employed in the disclosed methods of using.

As already mentioned, the present disclosure relates to a pharmaceutical composition comprising a therapeutically effective amount of a disclosed compound, a product of a disclosed method of making, a pharmaceutically acceptable salt, a hydrate thereof, a solvate thereof, a polymorph thereof, and a pharmaceutically acceptable carrier. Additionally, the present disclosure relates to a process for preparing such a pharmaceutical composition, characterized in that a pharmaceutically acceptable carrier is intimately mixed with a therapeutically effective amount of a compound according to the present disclosure.

### Methods of Using the Compounds

In another aspect, disclosed herein is method for treating one or more metabolic disorders, psychiatric disorders, neurological disorders, pain disorders, gastrointestinal disorders, cancers, inflammatory disorders, fibrotic disorders, liver diseases, cardiovascular disorders, chronic kidney disease, pulmonary conditions, a disease or condition associated with abnormal histone deacetylase activity, malaria-associated disorders, viral diseases, bacterial diseases, hair loss, substance overuse associated pathologies including, but not limited to, cannabinoid use disorder (CUD), alcohol use disorder (AUD), obesity-associated diseases, and/or skin conditions.

In one aspect, the metabolic disorder is obesity, diabetes, insulin resistance, diabetic nephropathy, obesity-induced chronic kidney disease or any combination thereof. In another aspect, the metabolic disorder can be a rare obesity-associated disease such as, for example, Prader-Willi Syndrome. In another aspect, the neurological disorder is Parkinson's disease, Alzheimer's disease, epilepsy, Huntington's disease, Tourette's syndrome, cerebral ischemia, or any combination thereof. In still another aspect, the pain disorder is low back pain, osteoarthritis, rheumatoid arthritis, headache, multiple sclerosis, fibromyalgia, shingles, neuropathy, endometriosis, or any combination thereof. In yet another aspect, the gastrointestinal disorder is Crohn's disease, ulcerative colitis, irritable bowel syndrome, celiac disease, gastrointestinal reflux disease, peptic ulcer disease, or any combination thereof. In one aspect, the cancer is non-Hodgkins lymphoma, neuroblastoma, sarcoma, metastatic brain cancers, ovarian cancer, prostate cancer, breast cancer, lymphoma, non-small cell lung carcinoma, gastric cancer, gastroesophageal junction adenocarcinoma, hematological cancers, melanoma, squamous cell carcinoma, Hodgkin's lymphoma, anaplastic large cell lymphoma, pancreatic cancer, acute lymphoblastic leukemia, acute myeloid leukemia, hepatocellular carcinoma, colorectal cancer, angiosarcoma, head and neck cancer, ovarian cancer, solid tumors, multiple myeloma, glioblastoma, testicular cancer, urothelial cancer, chronic lymphocytic leukemia, adenocortical carcinoma, acute myelogenous leukemia, clear cell renal cell carcinoma, chronic myelomonocytic leukemia, juvenile myelomonocytic leukemia, small cell lung carcinoma, hairy cell leukemia, renal cell carcinoma, nasopharyngeal cancer, glioma, chronic lymphatic leukemia, diffuse large B-cell lymphoma, gall bladder cancer, thyroid tumor, bone cancer, cervical cancer, uterine cancer, endometrial cancer, vulvar cancer, bladder cancer, colon cancer, colorectal cancer, pancreatic cancer, neuronal cancers, mesothelioma, cholangiocarcinoma, small bowel adenocarcinoma, epidermoid carcinoma, cancer of the pleural or peritoneal membranes, another cancer, or any combination thereof. In still another aspect, the inflammatory disorder is ankylosing spondylitis, gout, myositis, scleroderma, Sjogren's syndrome, asthma, lupus, vasculitis, or any combination thereof. In a further aspect, the liver disease is cirrhosis, fatty liver disease, or any combination thereof. In a still further aspect, the cardiovascular disorder is dyslipidemia, atherosclerosis, hypertension, cardiac fibrosis, cardiac hypertrophy, heart failure, or any combination thereof.

In any of these aspects, the subject can be a mammal. In another aspect, the mammal can be a cat, dog, rat, mouse, guinea pig, hamster, rabbit, horse, cattle, swine, sheep, goat, human, or another primate.

In one aspect, the disclosed method further include administering one or more additional drugs to the subject, wherein the one or more additional drugs can be selected from a GLP-1 agonist, an integrin inhibitor, a galectin inhibitor, a CB2 agonist, a CB1 antagonist, metformin, an AMPK activator, a mGlur5 antagonist, a 5HT2B antagonist, an iNOS inhibitor, a SSAO/VAP-1 inhibitor, or any combination thereof. In a further aspect, the compound or pharmaceutical composition and the one or more drugs can be administered simultaneously, or can be administered sequentially in any order.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Aspects

The present disclosure can be described in accordance with the following numbered aspects, which should not be confused with the claims.

Aspect 1. A compound comprising a structure of Formula I

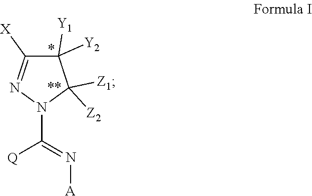

wherein X is naphthyl, heteroaryl, linear or branched C₁-C₁₀ alkyl, or

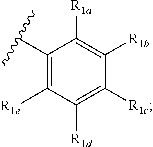

wherein each of R₁ₐ—R₁ₑ independently is selected from hydrogen, halogen, hydroxy, C₁-C₆ alkyl, —CF₃, —OCF₃, —O—(C₁-C₆ alkyl), —NO₂, or —CN;
wherein Y₁ and Y₂ independently are hydrogen, naphthyl, heteroaryl, linear or branched C₁-C₁₀ alkyl, or

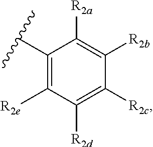

provided Y₁ and Y₂ are not both hydrogen;
wherein each of R₂ₐ—R₂ₑ independently is selected from hydrogen, halogen, hydroxy, C₁-C₆ alkyl, fluoroalkyl, fluoroalkoxy, —O—(C₁-C₆ alkyl), —NO₂, or —CN;
wherein Z₁ and Z₂ independently are hydrogen, —CO(═O)—(C₁-C₆ alkyl), or —C(═O)NH—(C₁-C₆ alkyl);
wherein Q is —S—R₃, —S(═O)—R₃, —S(═O)₂—R₃, —(CH₂)ₓ—R₃, —Se—R₃, —Se(═O)—R₃, or —Se(═O)₂—R₃, or OR₃;
wherein R₃ is selected from hydrogen, substituted or unsubstituted branched or linear C₁-C₆ alkyl, C₁-C₆ alkene, C₁-C₆ alkyne, C₁-C₆ allene, C₃-C₈ cycloalkyl, C₃-C₈ heterocycloalkyl, —(C(R₁₄ₐ)(R_{14b}))ₓ—C(═O)—N(R₁₃ₐ)(R_{13b}), —(C(R₁₄ₐ)(R_{14b}))_{y}—C(═O)—NHOH, or —(C(R₁₄ₐ)(R_{14b}))_{y}—CH(CH₃)—C(═O)(OR₁₀); —(CH₂)_{y}CN, —(CH₂)_{y}NO₂, —(CH₂)_{y}NHNH₂, —(C(R₁₄ₐ)(R_{14b}))_{y}SO₃R, —(C(R₁₄ₐ)(R_{14b}))_{y}N₃, —(C(R₁₄ₐ)(R_{14b}))_{y}—C₂H₂N₃, —(C(R₁₄ₐ)(R_{14b}))_{y}SON(R₁₃ₐ)(R_{13b}), —(C(R₁₄ₐ)(R_{14b}))_{y}SO₂N(R₁₃ₐ)(R_{13b}), —(C(R₁₄ₐ)(R_{14b}))_{y}PON(R₁₃ₐ)(R_{13b}), —(C(R₁₄ₐ)(R_{14b}))_{y}PO(OR₁₅)NR₁₆, —(C(R₁₄ₐ)(R_{14b}))_{y}B(OR₁₅)₃, —(C(R₁₄ₐ)(R_{14b}))_{y}(C═NR₁₆)N(R₁₃ₐ)(R_{13b}), C₁-C₆ alkoxy, OC(R₁₇)₃, OSi(R₁₇)₃, or —OCHF₂;
wherein x is from 1 to 6;
wherein y is from 0 to 6;
wherein R₁₀ is C₁-C₆ alkyl;
wherein each of R₁₃ₐ, R_{13b}, R₁₄ₐ, R_{14b}, R₁₅, R₁₆, and R₁₇, when present, is independently selected from hydrogen, halogen, hydroxy, C₁-C₆ alkyl, fluoroalkyl, fluoroalkoxy, —O—(C₁-C₆ alkyl), —NO₂, or —CN;
provided that when Q is —S—R₃, R₃ is not linear or branched alkyl;
wherein A is —SO₂—R₄, —SeO₂—R₄—S(═O)—R₄, —S(═O)(—R₄)(—R₅), —S(═O)(═NH)—R₄, —C(═O)—R₄, —Se—R₄, —Se(═O)—R₄, —Se(═O)(—R₄)(—R₅), or —N(R₁₉ₐ)(R_{19b});
wherein R₄ is

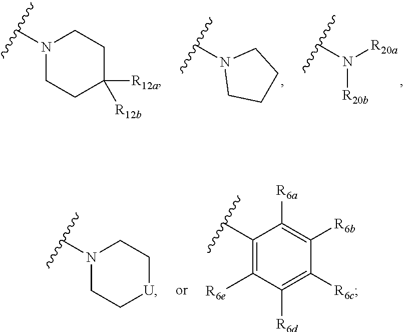

wherein each of R₁₉ₐ and R_{19b} is independently selected from hydrogen, halogen, hydroxy, substituted or unsubstituted C₁-C₆ alkyl, fluoroalkyl, fluoroalkoxy, —O—(C₁-C₆ alkyl), —NO₂, or —CN, and wherein R_{19b} is optionally absent;
wherein each of R₂₀ₐ and R_{20b} is independently selected from linear or branched C₁-C₆ alkyl or adamantyl;
   wherein each of R₁₂ₐ and R_{12b} independently is hydrogen, CF₃, phenyl, fluoro, or any combination thereof;
wherein U is NR₁₈, S, SO₂, or O;
   wherein R₁₈ is selected from hydrogen, halogen, hydroxy, C₁-C₆ alkyl, fluoroalkyl, fluoroalkoxy, —O—(C₁-C₆ alkyl), —NO₂, or —CN;
wherein each of R₆ₐ—R₆ₑ independently is selected from hydrogen, halogen, hydroxy, C₁-C₆ alkyl, fluoroalkyl, fluoroalkoxy, —O—(C₁-C₆ alkyl), —NO₂, —CN, —(CH₂)z-R₇, naphthyl, or heteroaryl;
wherein R₇ is halogen, —CN, fluoroalkyl, fluoroalkoxy, or alkoxy;
wherein z is from 1 to 3;
wherein R₅ is (═O), —NH—R₃, or

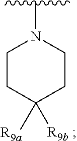

wherein R₃ is hydrogen or C₁-C₆ alkyl; and
wherein each of R₉ₐ and R_{9b} independently is hydrogen or halogen.

Aspect 2. The compound of aspect 1, wherein X is

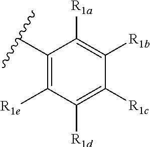

and each of R₁ₐ-R₁ₑis independently —CF₃, —OCF₃, or hydrogen.

Aspect 3. The compound of aspect 1, wherein X is

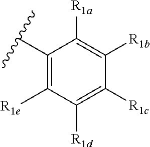

and each of R₁ₐ—R₁ₑ is independently hydrogen or halogen.

Aspect 4. The compound of aspect 1 or 3, wherein X is

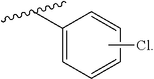

Aspect 5. The compound of aspect 4, wherein X is

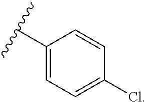 [SMILES: *c1ccc(Cl)cc1]

Aspect 6. The compound of aspect 1, wherein X is pyridyl.

Aspect 7. The compound of any one of aspects 1-6, wherein R₃ is —(CH₂)ₙ—R₁₁;
wherein n is from 1 to 6; and
wherein R₁₁ is cyano, methoxy, halogen, —CF₃, —OCF₃, nitro, or any combination thereof.

Aspect 8. The compound of aspect 7, wherein R₃ is —(CH₂)₃—CN, —(CH₂)₂—OCH₃, or any combination thereof.

Aspect 9. The compound of any one of aspects 1-8, wherein R₄ is

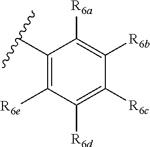

and each of R₆ₐ—R_{6c} is independently —CF₃, —OCF₃, or hydrogen.

Aspect 10. The compound of any one of aspects 1-9, wherein R₇ is —CF₃, —OCF₃, or methoxy.

Aspect 11. The compound of any one of aspects 1-10, wherein Y₁ is hydrogen and Y₂ is

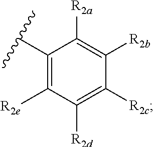

and
wherein each of R₂ₐ—R₂ₑ is independently hydrogen or halogen.

Aspect 12. The compound of aspect 11, wherein Y₂ is phenyl.

Aspect 13. The compound of any one of aspects 1-11, wherein Y₁, Y₂, or both are pyridyl.

Aspect 14. The compound of any one of aspects 1-13, having substantially (R) stereochemistry at a carbon indicated by *.

Aspect 15. The compound of any one of aspects 1-13, having substantially (S) stereochemistry at a carbon indicated by *.

Aspect 16. The compound of any one of aspects 1-15, wherein Z₁ and Z₂ are hydrogen.

Aspect 17. The compound of any one of aspects 1-15, having substantially (R) stereochemistry at a carbon indicated by **.

Aspect 18. The compound of any one of aspects 1-15, having substantially (S) stereochemistry at a carbon indicated by **.

Aspect 19. The compound of any one of aspects 1-18, wherein Q is selected from —S—R₃, —S(═O)—R₃, and —S(═O)₂—R₃.

Aspect 20. The compound of aspect 19, wherein R₃ is selected from

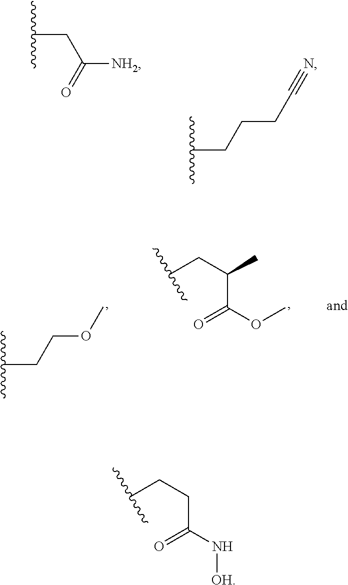

Aspect 21. The compound of any one of aspects 1-20, wherein A is —S—R₄, —S(═O)—R₄, or —S(═O)₂—R₄.

Aspect 22. The compound of aspect 21, wherein R₄ is selected from

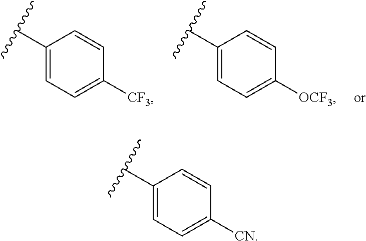 [SMILES: *c1ccc(C(F)(F)F)cc1 ; *c1ccc(OC(F)(F)F)cc1 ; *c1ccc(C#N)cc1]

Aspect 23. The compound of any one of aspects 1-22, wherein X is

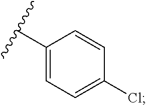

wherein Y₁ is hydrogen and Y₂ is phenyl;
wherein Z₁ and Z₂ are hydrogen;
wherein Q is selected from —S—R₃, —S(═O)—R₃, and —S(═O)₂—R₃ and R₃ is

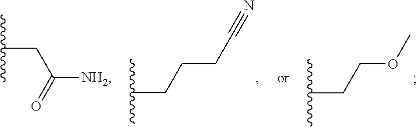

and
wherein A is —S—R₄, —S(═O)—R₄, or —S(═O)₂—R₄ and wherein R₄ is

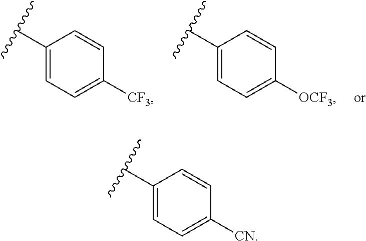 [SMILES: *c1ccc(C(F)(F)F)cc1 ; *c1ccc(OC(F)(F)F)cc1 ; *c1ccc(C#N)cc1]

Aspect 24. The compound of any one of aspects 1-23, wherein the compound has an inhibition constant for cannabinoid receptor 1 of less than about 100 nM.

Aspect 25. The compound of any one of aspects 1-23, wherein the compound has a inhibition constant for to cannabinoid receptor 1 of less than about 25 nM.

Aspect 26. A pharmaceutical composition comprising the compound of any one of aspects 1-25 or a pharmaceutically acceptable salt thereof.

Aspect 27. The pharmaceutical composition of aspect 26, further comprising at least one excipient.

Aspect 28. The pharmaceutical composition of aspect 26 or 27, wherein the pharmaceutical composition is administered to a subject orally, by inhalation, parenterally, intravenously, mucosally, or any combination thereof.

Aspect 29. A method for treating one or more metabolic disorders, psychiatric disorders, neurological disorders, pain disorders, gastrointestinal disorders, cancers, inflammatory disorders, fibrotic disorders, liver diseases, cardiovascular disorders, chronic kidney disease, pulmonary conditions, a disease or condition associated with abnormal histone deacetylase activity, malaria-associated disorders, viral diseases, bacterial diseases, hair loss, or substance overuse associated pathologies in a subject, the method comprising administering to the subject a therapeutically effective amount of the compound of any one of aspects 1-25 or the pharmaceutical composition of any one of aspects 26-28.

Aspect 30. The method of aspect 29, wherein the metabolic disorder is obesity, diabetes, insulin resistance, diabetic nephropathy, obesity-induced chronic kidney disease, Prader-Willi Syndrome, or any combination thereof.

Aspect 31. The method of aspect 29, wherein the neurological disorder is Parkinson's disease, Alzheimer's disease, epilepsy, Huntington's disease, Tourette's syndrome, cerebral ischemia, or any combination thereof.

Aspect 32. The method of aspect 29, wherein the pain disorder is low back pain, osteoarthritis, rheumatoid arthritis, headache, multiple sclerosis, fibromyalgia, shingles, neuropathy, endometriosis, or any combination thereof.

Aspect 33. The method of aspect 29, wherein the gastrointestinal disorder is Crohn's disease, ulcerative colitis, irritable bowel syndrome, celiac disease, gastrointestinal reflux disease, peptic ulcer disease, or any combination thereof.

Aspect 34. The method of aspect 29, wherein the cancer is non-Hodgkins lymphoma, neuroblastoma, sarcoma, metastatic brain cancers, ovarian cancer, prostate cancer, breast cancer, lymphoma, non-small cell lung carcinoma, gastric cancer, gastroesophageal junction adenocarcinoma, hematological cancers, melanoma, squamous cell carcinoma, Hodgkin's lymphoma, anaplastic large cell lymphoma, pancreatic cancer, acute lymphoblastic leukemia, acute myeloid leukemia, hepatocellular carcinoma, colorectal cancer, angiosarcoma, head and neck cancer, ovarian cancer, solid tumors, multiple myeloma, glioblastoma, testicular cancer, urothelial cancer, chronic lymphocytic leukemia, adenocortical carcinoma, acute myelogenous leukemia, clear cell renal cell carcinoma, chronic myelomonocytic leukemia, juvenile myelomonocytic leukemia, small cell lung carcinoma, hairy cell leukemia, renal cell carcinoma, nasopharyngeal cancer, glioma, chronic lymphatic leukemia, diffuse large B-cell lymphoma, gall bladder cancer, thyroid tumor, bone cancer, cervical cancer, uterine cancer, endometrial cancer, vulvar cancer, bladder cancer, colon cancer, colorectal cancer, pancreatic cancer, neuronal cancers, mesothelioma, cholangiocarcinoma, small bowel adenocarcinoma, epidermoid carcinoma, cancer of the pleural or peritoneal membranes, another cancer, or any combination thereof.

Aspect 35. The method of aspect 29, wherein the inflammatory disorder is ankylosing spondylitis, gout, myositis, scleroderma, Sjogren's syndrome, asthma, lupus, vasculitis, or any combination thereof.

Aspect 36. The method of aspect 29, wherein the liver disease is cirrhosis, alcoholic fatty liver disease, non-alcoholic fatty liver disease, or any combination thereof.

Aspect 37. The method of aspect 29, wherein the cardiovascular disorder is dyslipidemia, atherosclerosis, hypertension, cardiac fibrosis, cardiac hypertrophy, heart failure, or any combination thereof.

Aspect 38. The method of aspect 29, wherein the fibrotic disorder is idiopathic pulmonary fibrosis, Hermansky-Pudlak syndrome associated pulmonary fibrosis, radiation induced pulmonary fibrosis, scleroderma associated organ fibrosis, skin fibrosis, kidney fibrosis, liver fibrosis, chronic graft versus host disease (cGvHD), or any combination thereof.

Aspect 39. The method of aspect 29, wherein the substance overuse associated pathology is synthetic cannabinoid-induced acute organ toxicity, cannabinoid use disorder, alcohol use disorder, or any combination thereof.

Aspect 40. The method of any one of aspects 29-39, wherein the subject is a mammal.

Aspect 41. The method of aspect 40, wherein the mammal is a cat, dog, rat, mouse, guinea pig, hamster, rabbit, horse, cattle, swine, sheep, goat, human, or another primate.

Aspect 42. The method of any one of aspects 29-41, further comprising administering one or more additional drugs to the subject.

Aspect 43. The method of aspect 42, wherein the one or more additional drugs comprises a GLP-1 agonist, an integrin inhibitor, a galectin inhibitor, a CB2 agonist, a CB1 antagonist, metformin, an AMPK activator, a mGlur5 antagonist, a 5HT2B antagonist, an iNOS inhibitor, a SSAO/VAP-1 inhibitor, or any combination thereof.

Aspect 44. The method of aspect 42 or 43, wherein the compound or pharmaceutical composition and the one or more additional drugs are administered simultaneously.

Aspect 45. The method of aspect 42 or 43, wherein the compound or pharmaceutical composition and the one or more additional drugs are administered sequentially.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Exemplary Synthesis Methods

Exemplary methods for synthesizing the compounds disclosed herein are summarized in Scheme 1, with details being provided below and wherein X, Y₁, Y₂, Z₁, Z₂, R₃, and R₄ are defined as above.

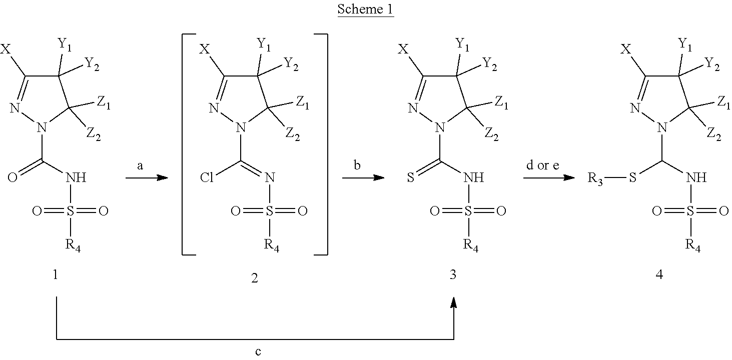

### Method 1

A first exemplary synthesis method consists of two steps. (a) In a first step, POCl₃ and base or PCl₅ are used to convert compound 1 to intermediate 2. (b) In a second step, Na₂S₂O₃ is used to convert intermediate 2 to compound 3.

### Method 2

A second exemplary synthesis method consists of a single step. (c) In this method, P₂S₅ or Lawesson reagent is used to convert compound 1 directly to compound 3.

### Functionalization of Thio Group

Following the synthesis of compound 3, the thio group can be modified to provide additional functionalities as in compound 4. (d or e) In a first functionalization method, R₃-LG can be used to contact compound 3 in refluxing methanol, where LG is a leaving group (e.g., Cl, Br, I, OTs, Oms, or the like) and R₃ is defined as above. In a second functionalization method, R₃-LG can be used to contact compound 3 in the presence of K₂CO₃ in DMF. Exemplary compounds synthesized and characterized include, but are not limited to, those shown in Table 1 and the exemplary structure below, wherein R_{6c} is defined as above and M and R are defined as in Table 1:

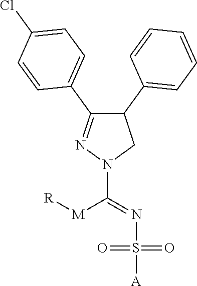

TABLE 1
Exemplary Compounds and Characterization Data

| Compound | | | | | LCMS |
|---|---|---|---|---|---|
| # | R | A | M | ¹H-NMR | (MH⁺) |
| 1 | 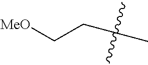 [SMILES: *CCOC] | 4-CF₃Ph | S | ¹H-NMR (800 MHz, CDCl₃): δ 8.09 (dd, J = 4.1, 2.0 Hz, 2H), 7.75-7.75 (m, 2H), 7.34 (s, 3H), 7.33-7.31 (m, 5H), 7.18-7.17 (m, 2H), 5.00-4.98 (m, 1H), 4.86-4.82 (m, 1H), 4.56-4.54 (m, 1H), 3.44 (dd, J = 1.8, 0.6 Hz, 2H), 3.21 (s, 3H), 3.08- 3.05 (m, 2H). | 582.1 |
| 2 | 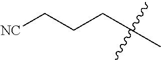 [SMILES: *CCCC#N] | 4-CF₃Ph | S | ¹H-NMR (800 MHz, CDCl₃): δ 8.07 (d, J = 7.5 Hz, 2H), 7.76 (d, J = 8.0 Hz, 2H), 7.59 (d, J = 7.6 Hz, 2H), 7.33 (d, J = 7.3 Hz, 2H), 7.27 (d, J = 8.5 Hz, 3H), 7.15 (d, J = 6.6 Hz, 2H), 4.98-4.97 (m, 1H), 4.84 (dd, J = 10.9, 5.3 Hz, 1H), 4.54 (td, J = 2.8, 1.2 Hz, 1H), 2.95 (t, J = 3.3 Hz, 2H), 2.26 (s, 2H), 1.86-1.86 (m, 2H). | 591.1 |
| 3 | 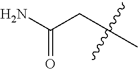 [SMILES: *CC(N)=O] | 4-CF₃Ph | S | ¹H-NMR (800 MHz, CDCl₃): δ 8.08-8.07 (m, 2H), 7.77-7.76 (m, 2H), 7.60-7.59 (m, 3H), 7.35 (d, J = 7.0 Hz, 4H), 7.29 (t, J = 4.3 Hz, 5H), 7.16- 7.15 (m, 2H), 6.61 (d, J = 9.4 Hz, 1H), 5.30 (s, 1H), 4.91-4.90 (m, 1H), 4.86-4.84 (m, 1H), 4.47- 4.46 (m, 1H), 3.73-3.71 (m, 1H), 3.66-3.64 (m, 1H). | 581.1 |
| 4 | 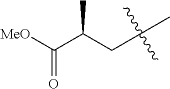 [SMILES: *C[C@H](C)C(=O)OC] | 4-CF₃Ph | S | ¹H-NMR (500 MHz, CDCl₃): δ 8.14 (d, J = 7.7 Hz, 2H), 7.81 (d, J = 8.1 Hz, 2H), 7.67-7.65 (m, 2H), 7.39 (t, J = 7.3 Hz, 2H), 7.35-7.32 (m, 2H), 7.23-7.21 (m, 2H), 5.13 (s, 1H), 4.90 (dt, J = 8.8, 3.5 Hz, 1H), 4.70-4.60 (m, 1H), 3.65 (s, 3H), 3.10 (dd, J = 13.8, 8.1 Hz, 1H), 2.88-2.83 (m, 1H), 2.69-2.67 (m, 1H), 1.00 (d, J = 7.1 Hz, 3H). | 624.1 |
| 5 | 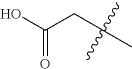 [SMILES: *CC(=O)O] | 4-CF₃Ph | S | ¹H-NMR (800 MHz, CDCl₃): δ 8.08-8.07 (m, 2H), 7.76-7.75 (m, 2H), 7.61-7.60 (m, 2H), 7.35 (d, J = 1.3 Hz, 2H), 7.29 (d, J = 1.5 Hz, 3H), 7.18 (t, J = 2.3 Hz, 2H), 5.12-5.10 (m, 1H), 4.91-4.89 (m, 1H), 4.68-4.67 (m, 1H), 3.59-3.57 (m, 2H). | 582.1 |
| 6 | 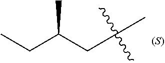 Mix of diastereomers | 4-CF₃Ph | S | ¹H-NMR (800 MHz, CDCl₃): δ 8.10-8.09 (m, 2H), 7.75-7.74 (m, 2H), 7.63-7.60 (m, 5H), 7.34- 7.28 (m, 6H), 7.17 (d, J = 3.2 Hz, 3H), 4.88-4.82 (m, 2H), 4.63-4.61 (m, 1H), 4.41-4.39 (m, 1H), 2.87-2.81 (m, 1H), 2.65-2.60 (m, 1H), 1.26-1.19 (m, 1H), 1.07-1.03 (m, 1H), 0.82-0.81 (m, 3H), 0.72-0.71 (m, 3H). | 594.10 |
| 7 | 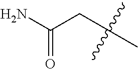 [SMILES: *CC(N)=O] | 4-OCF₃Ph | S | ¹H-NMR (800 MHz, CDCl₃): δ 8.00-7.99 (m, 2H), 7.60 (dd, J = 8.7, 2.1 Hz, 2H), 7.35-7.28 (m, 8H), 7.16-7.15 (m, 2H), 6.63 (s, 1H), 5.26 (s, 1H), 4.92-4.89 (m, 1H), 4.84 (d, J = 9.2 Hz, 1H), 4.46-4.45 (m, 1H), 3.74-3.71 (m, 1H), 3.67-3.64 (m, 1H). | 597.1 |
| 8 | 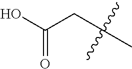 [SMILES: *CC(=O)O] | 4-OCF₃Ph | S | ¹H-NMR (800 MHz, CDCl₃): δ 7.98 (d, J = 8.7 Hz, 2H), 7.61 (d, J = 8.4 Hz, 2H), 7.34 (t, J = 7.5 Hz, 2H), 7.29 (dd, J = 13.3, 10.8 Hz, 5H), 7.18 (d, J = 7.3 Hz, 2H), 5.11 (t, J = 0.8 Hz, 1H), 4.90 (dd, J = 11.1, 5.6 Hz, 1H), 4.66-4.66 (m, 1H), 3.58 (q, J = 14.3 Hz, 2H). | 598.1 |
| 9 | 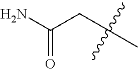 [SMILES: *CC(N)=O] | 4-CNPh | S | ¹H-NMR (800 MHz, CDCl₃): δ 8.05 (d, J = 7.7 Hz, 2H), 7.79 (d, J = 8.0 Hz, 2H), 7.60 (d, J = 8.2 Hz, 2H), 7.35 (t, J = 7.1 Hz, 3H), 7.30 (t, J = 6.1 Hz, 3H), 7.15 (d, J = 7.1 Hz, 2H), 6.58 (s, 1H), 5.34 (t, J = 1.4 Hz, 1H), 4.94-4.92 (m, 1H), 4.87- 4.85 (m, 1H), 4.47 (s, 1H), 3.70-3.68 (m, 1H), 3.62 (d, J = 16.3 Hz, 1H). | 538.1 |
| 10 | 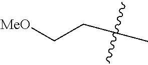 [SMILES: *CCOC] | 4-CNPh | S | ¹H-NMR (800 MHz, CDCl₃): δ 8.07 (d, J = 8.0 Hz, 2H), 7.77 (d, J = 8.4 Hz, 2H), 7.61 (d, J = 7.4 Hz, 2H), 7.33 (t, J = 7.5 Hz, 3H), 7.28 (d, J= 10.2 Hz, 4H), 7.16 (d, J = 7.4 Hz, 2H), 4.97-4.95 (m, 1H), 4.84-4.82 (m, 2H), 4.54-4.51 (m, 1H), 3.45 (t, J = 3.1 Hz, 2H), 3.24 (s, 3H), 3.04 (dt, J = 14.9, 6.4 Hz, 2H). | 539.1 |
| 11 | 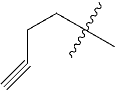 [SMILES: *CCC#C] | 4-CF₃Ph | Se | ¹H-NMR (800 MHz, CDCl₃): δ 8.07 (d, J = 7.7 Hz, 2H), 7.74 (d, J = 7.9 Hz, 2H), 7.57 (d, J = 7.6 Hz, 3H), 7.32 (s, 3H), 7.27 (s, 4H), 7.15 (d, J = 6.8 Hz, 3H), 5.01 (s, 1H), 4.88 (d, J = 5.0 Hz, 1H), 4.58 (d, J = 0.5 Hz, 1H), 4.10 (d, J = 7.0 Hz, 1H), 2.92 (d, J = 3.9 Hz, 2H), 2.45 (s, 2H). | 624.1 |
| 12 | Me | 4-CF₃Ph | Se | ¹H-NMR (800 MHz, CDCl₃): δ 8.07 (d, J = 7.9 | 586.1 |
| | | | | Hz, 2H), 7.72 (d, J = 7.9 Hz, 2H), 7.57 (d, J = 8.0 | |
| | | | | Hz, 2H), 7.31 (d, J = 7.1 Hz, 2H), 7.25 (t, J = 8.5 | |
| | | | | Hz, 3H), 7.13 (d, J = 7.5 Hz, 2H), 4.90 (t, J = 11.1 | |
| | | | | Hz, 1H), 4.82 (dd, J = 11.1, 5.2 Hz, 1H), 4.48 (d, | |
| | | | | J = 6.6 Hz, 1H), 2.25 (s, 3H). | |
| 13 | 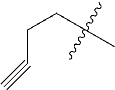 [SMILES: *CCC#C] | 4-CF₃Ph | S | ¹H-NMR (800 MHz, CDCl₃): δ 8.09 (d, J = 7.2 Hz, 2H), 7.76 (d, J = 7.1 Hz, 3H), 7.61 (d, J = 6.6 Hz, 3H), 7.34 (d, J = 6.7 Hz, 2H), 7.27 (dd, J = 9.2, 2.0 Hz, 4H), 7.17 (d, J = 6.5 Hz, 2H), 5.04 (s, 1H), 4.87-4.85 (m, 1H), 4.60 (s, 1H), 2.95 (d, J = 7.7 Hz, 2H), 2.39 (t, J = 3.2 Hz, 2H), 1.92 (d, J = 2.4 Hz, 1H). | 576.1 |
| 14 | 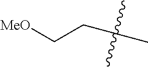 [SMILES: *CCOC] | 4-CF₃Ph | Se | H-NMR (800 MHz, CDCl₃): δ 8.09 (d, J = 7.4 Hz, 2H)), 7.75 (d, J = 7.3 Hz, 2H), 7.59 (d, J = 6.5 Hz, 2H), 7.34 (d, J = 6.8 Hz, 3H), 7.28-7.27 (m, 2H), 7.16 (d, J = 6.3 Hz, 2H), 4.97-4.96 (m, 1H), 4.87 (dd, J = 6.8, 3.9 Hz, 2H), 4.55-4.54 (m, 1H), 3.52 (dd, J = 6.4, 3.2 Hz, 2H), 3.21 (d, J = 3.1 Hz, 3H), 3.06 (dq, J = 9.3, 3.1 Hz, 3H). | 630.1 |
| 15 | Me | 4-ClPh | Se | H-NMR (800 MHz, CDCl₃): δ 7.88 (d, J = 8.4 Hz, | 551.9 |
| | | | | 2H), 7.57 (d, J = 8.4 Hz, 2H), 7.42 (d, J = 8.5 Hz, | |
| | | | | 2H), 7.31 (s, 2H), 7.25 (d, J = 3.8 Hz, 3H), 7.15 | |
| | | | | (dd, J = 30.8, 5.6 Hz, 2H), 4.89 (d, J = 11.4 Hz, | |
| | | | | 1H), 4.82 (dd, J = 11.2, 5.3 Hz, 1H), 4.47 (d, J = | |
| | | | | 6.9 Hz, 1H), 2.24 (s, 3H). | |
| 16 | 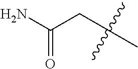 [SMILES: *CC(N)=O] | 4-CIPh | Se | ¹H-NMR (800 MHz, CDCl₃): δ 7.88 (d, J = 8.4 Hz, 2H), 7.58 (d, J = 8.5 Hz, 2H), 7.48 (d, J = 8.5 Hz, 2H), 7.35 (s, 2H), 7.28 (d, J = 8.5 Hz, 3H), 7.16 (s, 2H), 6.23 (s, 1H), 5.25 (s, 1H), 4.99 (t, J = 11.6 Hz, 1H), 4.90 (dd, J = 10.9, 5.3 Hz, 1H), 4.57-4.55 (m, 1H), 3.48 (s, 2H). | 594.9 |
| 17 | Me | 4-OCF₃Ph | Se | ¹H-NMR (800 MHz, CDCl₃): δ 7.94 (d, J = 7.8 | 601.1 |
| | | | | Hz, 2H), 7.52 (d, J = 7.9 Hz, 2H), 7.26-7.21 (m, | |
| | | | | 9H), 7.08 (d, J = 7.5 Hz, 2H), 4.85 (s, 1H), 4.77 | |
| | | | | (dd, J = 10.9, 5.2 Hz, 1H), 4.42 (d, J = 7.0 Hz, | |
| | | | | 1H), 2.20 (s, 3H). | |
| 18 | 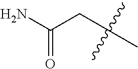 [SMILES: *CC(N)=O] | 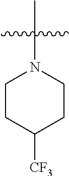 [SMILES: *N1CCC(C(F)(F)F)CC1] | S | ¹H-NMR (800 MHz, CDCl₃): δ 7.58 (d, J = 8.2 Hz, 2H), 7.33 (d, J = 7.4 Hz, 2H), 7.28 (d, J = 8.6 Hz, 3H), 7.15 (d, J = 7.3 Hz, 2H), 6.87 (s, 1H), 5.48 (s, 1H), 4.94-4.91 (m, 1H), 4.84 (dd, J = 11.2, 5.5 Hz, 1H), 4.46-4.45 (m, 1H), 3.79-3.67 (m, 4H), 2.78 (d, J = 6.8 Hz, 2H), 2.20 (s, 1H), 1.96 (d, J = 12.2 Hz, 2H), 1.70 (dd, J = 8.5, 4.2 Hz, 2H). | 588.1 |
| 19 | Me | 4-CF₃Ph | S | ¹H-NMR (800 MHz, CDCl₃): δ 8.10 (d, J = 8.1 | 538.1 |
| | | | | Hz, 2H), 7.74 (d, J = 8.1 Hz, 2H), 7.61 (d, J = 8.3 | |
| | | | | Hz, 2H), 7.33 (d, J = 7.4 Hz, 2H), 7.28 (t, J = 6.4 | |
| | | | | Hz, 3H), 7.17 (d, J = 7.4 Hz, 2H), 4.98-4.93 (m, | |
| | | | | 1H), 4.83 (dd, J = 11.1, 5.3 Hz, 1H), 4.54-4.51 | |
| | | | | (m, 1H), 2.34 (s, 3H). | |
| 20 | 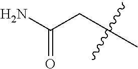 [SMILES: *CC(N)=O] | 4-CF₃Ph | Se | ¹H-NMR (800 MHz, CDCl₃): δ 8.08 (d, J = 8.0 Hz, 2H), 7.78 (d, J = 8.1 Hz, 2H), 7.58 (d, J = 8.1 Hz, 2H), 7.35 (d, J = 7.4 Hz, 2H), 7.30 (t, J = 6.2 Hz, 3H), 7.16 (d, J = 7.5 Hz, 2H), 6.23 (d, J = 0.6 Hz, 1H), 5.18 (s, 1H), 4.99 (t, J = 11.7 Hz, 1H), 4.91 (dt, J = 8.2, 4.0 Hz, 1H), 4.56-4.54 (m, 1H), 3.51 (s, 2H). | 629.1 |
| 21 | CH₂Cyp | 4-CF₃Ph | Se | ¹H-NMR (800 MHz, CDCl₃): δ 8.09 (d, J = 8.1 | 626.1 |
| | | | | Hz, 2H), 7.74 (d, J = 8.2 Hz, 2H), 7.58 (d, J = 8.2 | |
| | | | | Hz, 2H), 7.33 (t, J = 7.5 Hz, 3H), 7.28 (t, J = 9.3 | |
| | | | | Hz, 4H), 7.15 (d, J = 7.3 Hz, 2H), 4.87-4.82 (m, | |
| | | | | 2H), 4.45-4.43 (m, 1H), 2.96 (tt, J = 13.3, 7.1 Hz, | |
| | | | | 2H), 0.99-0.95 (m, 1H), 0.55 (d, J = 7.5 Hz, 2H), | |
| | | | | 0.16-0.14 (m, 2H). | |
| 22 | 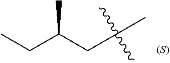 Mix of diastereomers | 4-ClPh | S | ¹H-NMR (800 MHz, CDCl₃): δ 7.90 (d, J = 8.2 Hz, 2H), 7.62 (d, J = 7.4 Hz, 2H), 7.44 (d, J = 8.5 Hz, 3H), 7.33 (t, J = 7.5 Hz, 3H), 7.31-7.27 (m, 3H), 7.17 (d, J = 6.9 Hz, 2H), 4.97 (s, 1H), 4.82 (dd, J = 10.8, 5.1 Hz, 1H), 4.52 (s, 1H), 2.89- 2.85 (m, 1H), 2.67-2.64 (m, 1H), 1.06 (dt, J = 10.7, 5.2 Hz, 1H), 0.84 (t, J = 3.2 Hz, 4H), 0.75 (d, J = 4.1 Hz, 4H). | 560.1 |
| 23 | 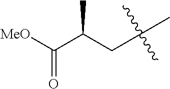 [SMILES: *C[C@H](C)C(=O)OC] (S) Mix of diastereomers | 4-ClPh | Se | ¹H-NMR (800 MHz, CDCl₃): δ 7.92 (d, J = 8.3 Hz, 2H), 7.60 (d, J = 7.8 Hz, 2H), 7.48 (d, J = 8.0 Hz, 2H), 7.35 (t, J = 7.4 Hz, 3H), 7.31-7.29 (m, 3H), 7.19 (d, J = 3.5 Hz, 2H), 5.07-5.06 (m, 1H), 4.89 (dt, J = 10.3, 4.9 Hz, 1H), 4.63-4.62 (m, 1H), 3.66 (d, J = 10.1 Hz, 3H), 3.04 (ddd, J = 18.6, 12.6, 8.2 Hz, 1H), 2.85 (dd, J = 12.7, 5.5 Hz, 1H), 2.71 (t, J = 7.1 Hz, 1H), 1.03 (s, 3H). | 638.1 |
| 24 | CH₂Cyp | 4-CNPh | Se | ¹H-NMR (800 MHz, CDCl₃): δ 8.07 (d, J = 8.0 | 583.1 |
| | | | | Hz, 2H), 7.77 (d, J = 8.2 Hz, 2H), 7.58 (d, J = 7.7 | |
| | | | | Hz, 2H), 7.33 (d, J = 7.4 Hz, 2H), 7.28 (t, J = 6.6 | |
| | | | | Hz, 3H), 7.14 (d, J = 7.1 Hz, 2H), 4.86-4.82 (m, | |
| | | | | 2H), 4.41 (s, 1H), 2.95 (d, J = 6.8 Hz, 2H), 0.98 | |
| | | | | (ddd, J = 7.6, 4.5, 3.1 Hz, 1H), 0.56 (d, J = 7.6 | |
| | | | | Hz, 2H), 0.17 (d, J = 4.3 Hz, 2H). | |
| 25 | 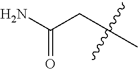 [SMILES: *CC(N)=O] | 4-CNPh | Se | ¹H-NMR (800 MHz, CDCl₃): δ 8.06 (d, J = 8.1 Hz, 2H), 7.81 (d, J = 8.2 Hz, 2H), 7.58 (d, J = 8.4 Hz, 2H), 7.35 (d, J = 7.4 Hz, 2H), 7.30 (d, J= 6.3 Hz, 4H), 7.16 (d, J = 7.1 Hz, 3H), 6.27-6.24 (m, 1H), 5.24-5.20 (m, 1H), 4.98-4.92 (m, 2H), 4.53- 4.51 (m, 1H), 3.51 (s, 2H). | 586.1 |
| 26 | 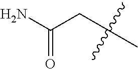 [SMILES: *CC(N)=O] | 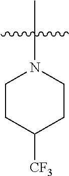 [SMILES: *N1CCC(C(F)(F)F)CC1] | Se | ¹H-NMR (800 MHz, CDCl₃): δ 7.56 (d, J = 8.4 Hz, 2H), 7.33 (d, J = 7.5 Hz, 3H), 7.28 (t, J = 8.7 Hz, 4H), 7.15 (d, J = 7.2 Hz, 2H), 6.71 (s, 1H), 5.57 (s, 1H), 4.94 (d, J = 11.8 Hz, 1H), 4.87 (dd, J= 11.2, 5.5 Hz, 1H), 4.49 (dd, J = 12.0, 5.3 Hz, 1H), 3.81 (s, 2H), 3.66 (d, J = 4.4 Hz, 2H), 2.78 (t, J = 12.0 Hz, 2H), 2.20 (s, 1H), 1.97 (d, J = 12.6 Hz, 2H), 1.71-1.69 (m, 3H). | 636.1 |

Additional synthesized compounds are listed by IUPAC name and mass (LCMS [M+H]) include, but are not limited to, those listed in Table 2 below:

TABLE 2
Additional Compounds and Masses

| | LCMS |
|---|---|
| IUPAC Name | (MH⁺) |
| 2-Hydroxyethyl (Z)-3-(4-chlorophenyl)-4-phenyl-N-((4-(trifluoromethyl)phenyl)sulfonyl)-4,5-dihydro-1H- | 568.1 |
| pyrazole-1-carbimidothioate | |
| 2-Hydroxyethyl (Z)-3-(4-chlorophenyl)-4-phenyl-N-((4-(trifluoromethyl)phenyl)sulfonyl)-4,5-dihydro-1H- | 615.1 |
| pyrazole-1-carbimidoselenoate | |
| N-(-(3-(4-chlorophenyl)-4-phenyl-4,5-dihydro-1H-pyrazol-1-yl)((R)-(2- | 598.1 |
| methoxyethyl)sulfinyl)methylene)-4-(trifluoromethyl)benzenesulfonamide | |
| N-((3-(4-chlorophenyl)-4-phenyl-4,5-dihydro-1H-pyrazol-1-yl)((2-methoxyethyl)sulfonyl)methylene)-4- | 614.1 |
| (trifluoromethyl)benzenesulfonamide | |
| N-(-(3-(4-chlorophenyl)-4-phenyl-4,5-dihydro-1H-pyrazol-1-yl)((R)-methylsulfinyl)methylene)-4- | 554.1 |
| (trifluoromethyl)benzenesulfonamide | |
| N-((3-(4-chlorophenyl)-4-phenyl-4,5-dihydro-1H-pyrazol-1-yl)(methylsulfonyl)methylene)-4- | 570.1 |
| (trifluoromethyl)benzenesulfonamide | |
| (Tetrahydro-2H-pyran-4-yl)methyl (Z)-3-(4-chlorophenyl)-4-phenyl-N-((4- | 622.1 |
| (trifluoromethyl)phenyl)sulfonyl)-4,5-dihydro-1H-pyrazole-1-carbimidothioate | |
| 3-Cyanopropyl (Z)-3-(4-chlorophenyl)-4-phenyl-N-((4-(trifluoromethoxy)phenyl)sulfonyl)-4,5-dihydro- | 607.1 |
| 1H-pyrazole-1-carbimidothioate | |
| N-((Z)-(3-(4-chlorophenyl)-4-phenyl-4,5-dihydro-1H-pyrazol-1-yl)((R)-(3- | 623.1 |
| cyanopropyl)sulfinyl)methylene)-4-(trifluoromethoxy)benzenesulfonamide | |
| 6,6-Dimethyl-5,6-dihydroimidazo[2,1-b]thiazol-3-yl)methyl (Z)-3-(4-chlorophenyl)-4-phenyl-N-((4- | 690.2 |
| (trifluoromethyl)phenyl)sulfonyl)-4,5-dihydro-1H-pyrazole-1-carbimidothioate | |
| (2-(((3-(4-chlorophenyl)-4-phenyl-4,5-dihydro-1H-pyrazol-1-yl) (((4- | 667.1 |
| (trifluoromethyl)phenyl)sulfonyl)imino)methyl)thio)acetyl)alanine | |
| Methyl (2-(((3-(4-chlorophenyl)-4-phenyl-4,5-dihydro-1H-pyrazol-1-yl)(((4- | 695.1 |
| (trifluoromethyl)phenyl)sulfonyl)imino) methyl)thio)acetyl)valinate | |
| 2-Oxo-2-((1-phenylethyl)amino)ethyl (Z)-3-(4-chlorophenyl)-4-phenyl-N-((4- | 685.1 |
| (trifluoromethyl)phenyl)sulfonyl)-4,5-dihydro-1H-pyrazole-1-carbimidothioate | |
| (2-Methylbutyl (Z)-3-(4-chlorophenyl)-N-((4-chlorophenyl)sulfonyl)-4-phenyl-4,5-dihydro-1H-pyrazole- | 640.0 |
| 1-carbimidoselenoate (mix of diastereomers) | |
| Methyl (3-(((3-(4-chlorophenyl)-4-phenyl-4,5-dihydro-1H-pyrazol-1-yl)(((4- | 672.0 |
| (trifluoromethyl)phenyl)sulfonyl)imino)methyl)selanyl)-2-methylpropanoate (mix of diastereomers) | |
| 2-(((3-(4-chlorophenyl)-4-phenyl-4,5-dihydro-1H-pyrazol-1-yl)(((4-(trifluoromethyl)piperidin-1- | 637.1 |
| yl)sulfonyl)imino)methyl)selanyl)acetic acid | |
| 2-(((3-(4-chlorophenyl)-4-phenyl-4,5-dihydro-1H-pyrazol-1-yl)(((4-(trifluoromethyl)piperidin-1- | |
| yl)sulfonyl)imino) methyl)thio)acetic acid | |
| Methyl (3-(4-chlorophenyl)-4-phenyl-N-((4-(trifluoromethyl)piperidin-1-yl)sulfonyl)-4,5-dihydro-1H- | |
| pyrazole-1-carbimidoselenoate | |
| 2-Amino-2-oxoethyl 3-(4-chlorophenyl)-4-phenyl-N-(piperidin-1-ylsulfonyl)-4,5-dihydro-1H-pyrazole-1- | |
| carbimidoselenoate | |
| 2-Amino-2-oxoethyl 3-(4-chlorophenyl)-4-phenyl-N-(piperidin-1-ylsulfonyl)-4,5-dihydro-1H-pyrazole-1- | |
| carbimidothioate | |
| 2-Amino-2-oxoethyl (3-(4-chlorophenyl)-N-(N,N-diethylsulfamoyl)-4-phenyl-4,5-dihydro-1H-pyrazole-1- | |
| carbimidothioate | |
| 2-Amino-2-oxoethyl (3-(4-chlorophenyl)-N-(N,N-diethylsulfamoyl)-4-phenyl-4,5-dihydro-1H-pyrazole-1- | 556.1 |
| carbimidoselenoate | |
| 2-(((3-(4-chlorophenyl)-4-phenyl-4,5-dihydro-1H-pyrazol-1-yl)((N,N- | |
| diethylsulfamoyl)imino)methyl)selanyl)acetic acid | |
| 2-(((3-(4-chlorophenyl)-4-phenyl-4,5-dihydro-1H-pyrazol-1-yl)((N,N- | |
| diethylsulfamoyl)imino)methyl)selanyl)acetic acid | |
| 2-Hydroxyethyl (3-(4-chlorophenyl)-4-phenyl-N-(piperidin-1-ylsulfonyl)-4,5-dihydro-1H-pyrazole-1- | |
| carbimidothioate | |
| 2-Hydroxyethyl (S,Z)-3-(4-chlorophenyl)-4-phenyl-N-((4-(trifluoromethyl)piperidin-1-yl)sulfonyl)-4,5- | |
| dihydro-1H-pyrazole-1-carbimidothioate | |
| 2-Amino-2-oxoethyl (S,Z)-3-(4-chlorophenyl)-N-((4,4-difluoropiperidin-1-yl)sulfonyl)-4-phenyl-4,5- | |
| dihydro-1H-pyrazole-1-carbimidothioate | |
| 3-(4-chlorophenyl)-4-phenyl-N-((4-(trifluoromethyl)piperidin-1-yl)sulfonyl)-4,5-dihydro-1H-pyrazole-1- | |
| carbimidic cyanic selenoanhydride | |
| 3-(4-chlorophenyl)-4-phenyl-N-((4-(trifluoromethyl)phenyl)sulfonyl)-4,5-dihydro-1H-pyrazole-1- | |
| carbimidic cyanic thioanhydride | |
| 3-(4-chlorophenyl)-4-phenyl-N-((4-(trifluoromethyl)piperidin-1-yl)sulfonyl)-4,5-dihydro-1H-pyrazole-1- | |
| carbimidic cyanic thioanhydride | |
| 3-(4-chlorophenyl)-4-phenyl-N-(piperidin-1-ylsulfonyl)-4,5-dihydro-1H-pyrazole-1-carbimidic cyanic | |
| thioanhydride | |
| 2-(((3-(4-chlorophenyl)-4-phenyl-4,5-dihydro-1H-pyrazol-1-yl)(((4-cyanopiperidin-1- | |
| yl)sulfonyl)imino)methyl)selanyl)acetic acid | |
| 2-hydroxyethyl-3-(4-chlorophenyl)-N-((4-cyanopiperidin-1-yl)sulfonyl)-4-phenyl-4,5-dihydro- | 593.1 |
| 1H-pyrazole-1-carbimidoselenoate | |
| 2-amino-2-oxoethyl-3-(4-chlorophenyl)-N-((4-cyanopiperidin-1-yl)sulfonyl)-4-phenyl-4,5- | |
| dihydro-1H-pyrazole-1-carbimidoselenoate | |

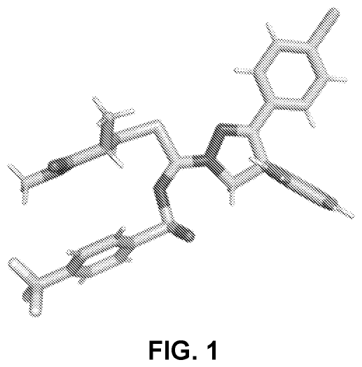

An exemplary three-dimensional structure of a disclosed compound is shown in FIG. 1.

### Example 2: Additional Synthetic Procedures

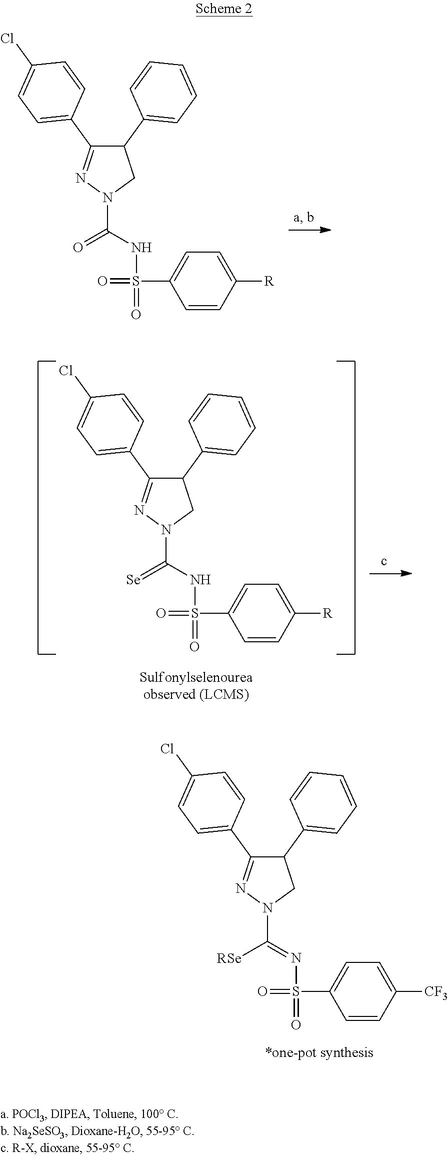

A general procedure for producing sulfonylselenoureas is shown in Scheme 2. By using sulfur containing salts in step (b) (e.g. Na₂S₂SO₃), analogous thiosulfonylureas can be produced.

General Procedure for One-pot Thiosulfonylurea formation and Thioalkylations

To a mixture of sulfonylurea compound (1eq) in toluene (8 mL), POCl₃ (1.5 eq), was added, followed by the addition of N, N-diisopropylethylamine (DIPEA) (1.5 eq) and the mixture was heated to 95° C. for 1.5 h under N₂ atmosphere. The reaction mixture was then cooled, and the excess reagents in toluene were evaporated in vacuo. The imidoyl chloride intermediate was dissolved in methanol (10 ml), to this solution was added dropwise, sodium thiosulfate (2 eq, dissolved in 0.5 ml water and the reaction was heated to 90° C. for 1h. Upon completion of reaction as seen by TLC/LCMS, alkylating agent was added drop-wise to the reaction mixture and reaction continued until all the thiourea is consumed as seen by TLC/LCMS. The reaction was cooled to room temperature and methanol was evaporated. The organic mixture was the extracted into dichloromethane, washed with brine, and dried over Na₂SO₄. The solvent was removed in vacuo and dried thoroughly to afford a sticky solid. The sticky solid was purified by flash chromatography (20% hexanes in EtOAc) to give thio-alkylated sulfonyl compounds, General yields ranged from 20-80%. Alternatively, the imidoyl chloride intermediate was dissolved in methanol (10 ml), to this solution was added dropwise, sodium thiosulfate (5 eq, dissolved in 0.5 ml water and the reaction was heated to 55-60° C. for over 3 h. Upon completion of reaction as seen by TLC/LCMS, alkylating agent was added drop-wise to the reaction mixture and reaction continued at the same temperature until all the thiourea is consumed as seen by TLC/LCMS. The reaction was cooled to room temperature and methanol was evaporated.

### General Procedure for One-Pot Sulfonylselenylurea Formation and Alkylation

To a mixture of sulfonylurea compound (1 eq) in toluene (5 mL), POCl₃ (1.5 mmol), was added, followed by the addition of N, N-diisopropylethylamine (DIPEA) (1.5 mmol) and the mixture was heated to 95° C. for 1.5 h under N₂ atmosphere. The reaction mixture was then cooled, and the excess reagents in toluene were evaporated in vacuo. The imidoyl chloride intermediate was dissolved in dioxane (10 mL), to this solution was added dropwise, premade aq. Na₂SeSO₃ (2 eq) and the reaction was heated to 90° C. Upon consumption of chloro-intermediate (TLC/LCMS), alkylating agent was added dropwise to the reaction mixture reaction continued until all the ‘selenosulfonylurea’ was consumed. The reaction was cooled to room temperature and dioxane was evaporated. The organic mixture was the extracted into dichloromethane, washed with brine, and dried over Na₂SO₄. The solvent was removed in vacuo and dried thoroughly to afford seleno-compounds. The resultant solid was purified by flash chromatography (20% hexanes in EtOAc) to yield novel seleno-compounds as powders. General yields ranged from 20-60%.

Alternatively, the imidoyl chloride intermediate was dissolved in dioxane (10 mL), to this solution was added dropwise, premade aq. Na₂SeSO₃ (5 eq) and the reaction was heated at 55-60° C. for over 3 h. Upon completion of reaction as seen by TLC/LCMS, alkylating agent was added drop-wise to the reaction mixture and reaction continued at the same temperature until all the ‘selenosulfonylurea’ was consumed as seen by TLC/LCMS. The reaction was cooled to room temperature and worked up as above.

### Example 3: Biological Data

TABLE 3
Exemplary Cannabinoid Receptor
Modulator Compounds and Activity

| | |
|---|---|
| 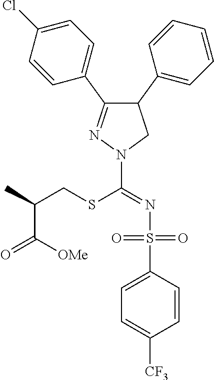 [SMILES: COC(=O)[C@@H](C)CS/C(=N\S(=O)(=O)c1ccc(C(F)(F)F)cc1)N1CC(c2ccccc2)C(c2ccc(Cl)cc2)=N1] | 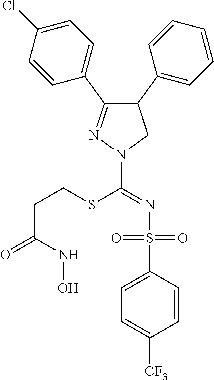 |
| Compound 3A | Compound 3B (CB1 |
| (CB1 Kᵢ = 7.7 nM) | antagonist, potential |
| | histone deacetylase |
| | inhibitor) |

In general Se-augmented compounds may show similar or better CB1 binding affinity than S-augmented compounds. For example, compounds 12 and 19 (see Table 1) have CB1R Kᵢ=3.3 nM and 16.6 nM, respectively. Similarly, compounds 20 and 3 have CB1R Kᵢ=14.4 nM and 21.2 nM, respectively. Additional cannabinoid receptor 1 (CB1R) inhibition constants for exemplary compounds is presented in Table 4:

TABLE 4
CB1R Kᵢ for Selected Compounds

| | CB1R |
|---|---|
| Compound | Ki |
| (See Table 1) | (nM) |
| 21 | 12 |
| 1 | 6.9 |
| 2 | 3.4 |
| 5 | 63 |
| 23 | 12.1 |

Additional In vitro characterization data for exemplary compounds is provided in Table 5 below (see Tables 1 and 3 for compound structures):

TABLE 5
Additional in vitro Characterization Data for Exemplary Compounds

| | | Mean | Mean | Mean | | |
|---|---|---|---|---|---|---|
| | Caco-2 | Pₐₚₚ | Pₐₚₚ | (B-A/ | | β- |
| | Assay | A-B | B-A | A-B) | cAMP | Arrestin |
| | Conc | (10⁻⁶ | (10⁻⁶ | Efflux | IC₅₀ | IC₅₀ |
| Compound ID | μM | cm/s) | cm/s) | Ratio | (μM) | (μM) |
| Compound 2 | 10 | 0.005610 | 0.0118 | 2.09 | 2.49 | 1.8 |
| Compound 3A | 10 | 0.009600 | 0.0121 | 1.26 | 0.00061 | 0.00004 |
| Compound 20 | 10 | 0.183 | 0.0451 | 0.25 | — | — |
| Compound 24 | — | — | — | — | 0.056 | 0.114 |
| Compound 21 | — | — | — | — | 0.023 | 0.028 |
| Compound 10 | — | — | — | — | 1.69 | 1.16 |

CB1 antagonism assays were conducted on red (cAMP) and green (R-Arrestin) NOMAD biosensors using MPXNomad-CB1 HEK293 cells using commercial kits and protocols provided by Innoprot (Derio, Spain).

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Iyer, M. R. et al., One-Pot Synthesis of Thio-Augmented Sulfonyl Ureas via a Modified Bunte's Reaction, ACS Omega, 2022, 35:31612-31620.

1. A compound comprising a structure of Formula I 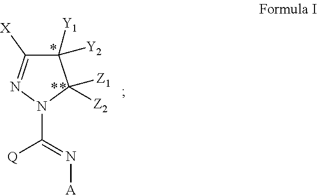
wherein X is naphthyl, heteroaryl, linear or branched C₁-C₁₀ alkyl, or 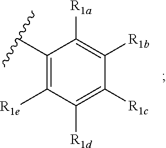
wherein each of R₁ₐ—R₁ₑ independently is selected from hydrogen, halogen, hydroxy, C₁-C₆ alkyl, —CF₃, —OCF₃, —O—(C₁-C₆ alkyl), —NO₂, or —CN;
wherein Y₁ and Y₂ independently are hydrogen, naphthyl, heteroaryl, linear or branched C₁-C₁₀ alkyl, or 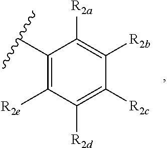
provided Y₁ and Y₂ are not both hydrogen;
wherein each of R₂ₐ—R₂ₑ independently is selected from hydrogen, halogen, hydroxy, C₁-C₆ alkyl, fluoroalkyl, fluoroalkoxy, —O—(C₁-C₆ alkyl), —NO₂, or —CN;
wherein Z₁ and Z₂ independently are hydrogen, —CO(═O)—(C₁-C₆ alkyl), or —C(═O)NH—(C₁-C₆ alkyl);
wherein Q is —S—R₃, —S(═O)—R₃, —S(═O)₂—R₃, —(CH₂)ₓ—R₃, —Se—R₃, —Se(═O)—R₃, or —Se(═O)₂—R₃, or OR₃;
wherein R₃ is selected from hydrogen, substituted or unsubstituted branched or linear C₁-C₆ alkyl, C₁-C₆ alkene, C₁-C₆ alkyne, C₁-C₆ allene, C₃-C₈ cycloalkyl, C₃-C₈ heterocycloalkyl, —(C(R₁₄ₐ)(R_{14b}))ₓ—C(═O)—N(R₁₃ₐ)(R_{13b}), —(C(R₁₄ₐ)(R_{14b}))_{y}—C(═O)—NHOH, or —(C(R₁₄ₐ)(R_{14b}))_{y}—CH(CH₃)—C(═O)(OR₁₀); —(CH₂)_{y}CN, —(CH₂)_{y}NO₂, —(CH₂)_{y}NHNH₂, —(C(R₁₄ₐ)(R_{14b}))_{y}SO₃R, —(C(R₁₄ₐ)(R_{14b}))_{y}N₃, —(C(R₁₄ₐ)(R_{14b}))_{y}—C₂H₂N₃, —(C(R₁₄ₐ)(R_{14b}))_{y}SON(R₁₃ₐ)(R_{13b}), —(C(R₁₄ₐ)(R_{14b}))_{y}SO₂N(R₁₃ₐ)(R_{13b}), —(C(R₁₄ₐ)(R_{14b}))_{y}PON(R₁₃ₐ)(R_{13b}), —(C(R₁₄ₐ)(R_{14b}))_{y}PO(OR₁₅)NR₁₆, —(C(R₁₄ₐ)(R_{14b}))_{y}B(OR₁₅)₃, —(C(R₁₄ₐ)(R_{14b}))_{y}(C═NR₁₆)N(R₁₃ₐ)(R_{13b}), C₁-C₆ alkoxy, OC(R₁₇)₃, OSi(R₁₇)₃, or —OCHF₂;
wherein x is from 1 to 6;
wherein y is from 0 to 6;
wherein R₁₀ is C₁-C₆ alkyl;
wherein each of R₁₃ₐ, R_{13b}, R₁₄ₐ, R_{14b}, R₁₅, R₁₆, and R₁₇, when present, is independently selected from hydrogen, halogen, hydroxy, C₁-C₆ alkyl, fluoroalkyl, fluoroalkoxy, —O—(C₁-C₆ alkyl), —NO₂, or —CN;
provided that when Q is —S—R₃, R₃ is not linear or branched alkyl;
wherein A is —SO₂—R₄, —SeO₂—R₄—S(═O)—R₄, —S(═O)(—R₄)(—R₅), —S(═O)(═NH)—R₄, —C(═O)—R₄, —Se—R₄, —Se(═O)—R₄, —Se(═O)(—R₄)(—R₅), or —N(R₁₉ₐ)(R_{19b});
wherein R₄ is 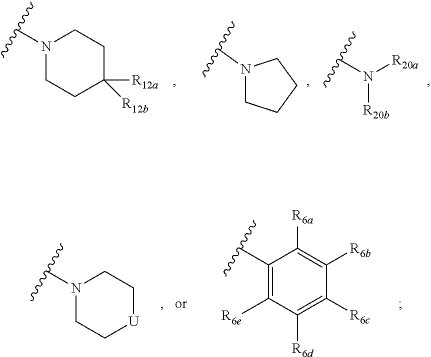
wherein each of R₁₉ₐ and R_{19b} is independently selected from hydrogen, halogen, hydroxy, substituted or unsubstituted C₁-C₆ alkyl, fluoroalkyl, fluoroalkoxy, —O—(C₁-C₆ alkyl), —NO₂, or —CN, and wherein R_{19b} is optionally absent;
wherein each of R₂₀ₐ and R_{20b} is independently selected from linear or branched C₁-C₆ alkyl or adamantyl;
wherein each of R₁₂ₐ and R_{12b} independently is hydrogen, CF₃, phenyl, fluoro, or any combination thereof;
wherein U is NR₁₃, S, SO₂, or O;
wherein R₁₈ is selected from hydrogen, halogen, hydroxy, C₁-C₆ alkyl, fluoroalkyl, fluoroalkoxy, —O—(C₁-C₆ alkyl), —NO₂, or —CN;
wherein each of Rea-R₆ₑ independently is selected from hydrogen, halogen, hydroxy, C₁-C₆ alkyl, fluoroalkyl, fluoroalkoxy, —O—(C₁-C₆ alkyl), —NO₂, —CN, —(CH₂)z-R₇, naphthyl, or heteroaryl;
wherein R₇ is halogen, —CN, fluoroalkyl, fluoroalkoxy, or alkoxy;
wherein z is from 1 to 3;
wherein R₅ is (═O), —NH—R₈, or 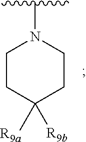
wherein R₈ is hydrogen or C₁-C₆ alkyl; and
wherein each of R₉ₐ and R_{9b} independently is hydrogen or halogen.

2. The compound of claim 1, wherein X is 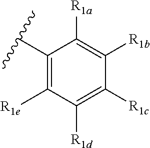
and each of R₁ₐ—R₁ₑ is independently —CF₃, —OCF₃, or hydrogen.

3. The compound of claim 1, wherein X is 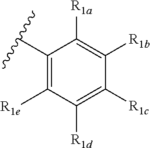
and each of R₁ₐ—R₁ₑ is independently hydrogen or halogen.

4. (canceled)

5. The compound of claim 3, wherein X is

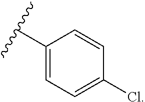 [SMILES: *c1ccc(Cl)cc1]

6. The compound of claim 1, wherein X is pyridyl.

7. The compound of claim 1, wherein R₃ is —(CH₂)ₙ—R₁₁;
wherein n is from 1 to 6; and
wherein R₁₁ is cyano, methoxy, halogen, —CF₃, —OCF₃, nitro, or any combination thereof.

8. The compound of claim 7, wherein R₃ is —(CH₂)₃—CN, —(CH₂)₂—OCH₃, or any combination thereof.

9. The compound of claim 1, wherein R₄ is 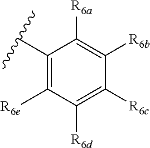
and each of R₆ₐ—R_{6c} is independently —CF₃, —OCF₃, or hydrogen.

10. (canceled)

11. The compound of claim 1, wherein Y₁ is hydrogen and Y₂ is 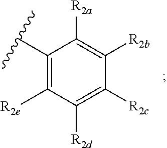
and
wherein each of R₂ₐ—R₂ₑ is independently hydrogen or halogen.

12. The compound of claim 11, wherein Y₂ is phenyl.

13. The compound of claim 1, wherein Y₁, Y₂, or both are pyridyl.

14. The compound of claim 1, having substantially (R) stereochemistry at a carbon indicated by *.

15. The compound of claim 1, having substantially (S) stereochemistry at a carbon indicated by *.

16. (canceled)

17. The compound of claim 1, having substantially (R) stereochemistry at a carbon indicated by **.

18. The compound of claim 1, having substantially (S) stereochemistry at a carbon indicated by **.

19. The compound of claim 1, wherein Q is selected from —S—R₃, —S(═O)—R₃, and —S(═O)₂—R₃.

20. The compound of claim 19, wherein R₃ is selected from

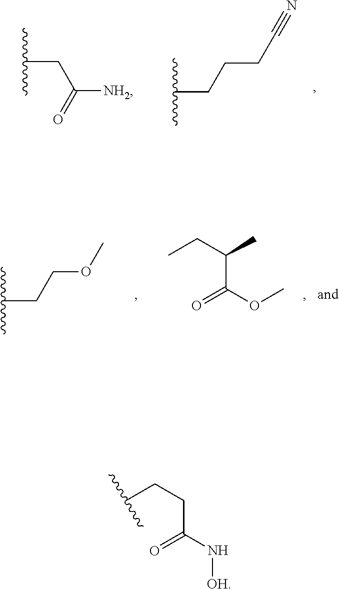

21. The compound of claim 1, wherein A is —S—R₄, —S(═O)—R₄, or —S(═O)₂—R₄.

22. The compound of claim 21, wherein R₄ is selected from

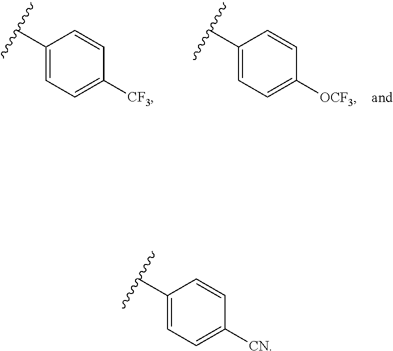

23. The compound of claim 1, wherein X is 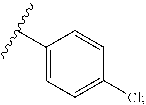
wherein Y₁ is hydrogen and Y₂ is phenyl;
wherein Z₁ and Z₂ are hydrogen;
wherein Q is selected from —S—R₃, —S(═O)—R₃, and —S(═O)₂—R₃ and R₃ is 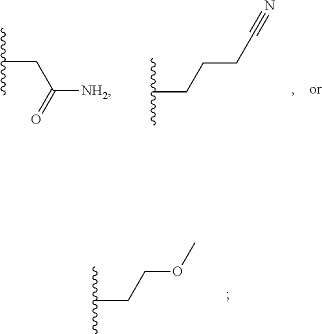
and
wherein A is —S—R₄, —S(═O)—R₄, or —S(═O)₂—R₄ and wherein R₄ is

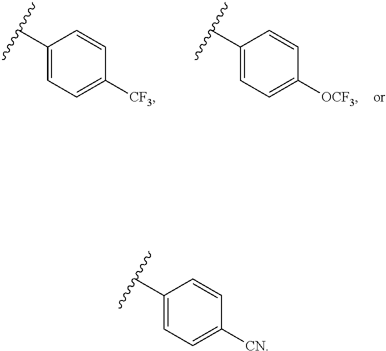

24.-45. (canceled)
